SEQUENCE LISTING (20 sequences)

SEQ ID NO: 1 - DNA, 7082 bp, Homo sapiens
  CDS (16)..(2916): CNGH0002.1
gtgtcgctcgctttcatgcagcctctctccctctccctctcccctctccttcctctcgcttcctctctcgcacctgagcgtacgcacctgcccgggcccggctccctcctcctctcccctccctctttccccgcccggccgcgggagcctcgtggctgcgtcaccgccgcccccccagacaagatggacaccgcggaggaagacatatgtagagtgtgtcggtcagaaggaacacctgagaaaccgctttatcatccttgtgtatgtactggcagtattaagtttatccatcaagaatgcttagttcaatggctgaaacacagtcgaaaagaatactgtgaattatgcaagcacagatttgcttttacaccaatttattctccagatatgccttcacggcttccaattcaagacatatttgctggactggttacaagtattggcactgcaatacgatattggtttcattatacacttgtggcctttgcatggttgggagttgttcctcttacagcatgccgcatctacaagtgcttgtttactggctccgtgagctcactactgacgctgccattagatatgctgtcaacggaaaatttgttggcagattgtttgcagggttgttttgtggtgacgtgcacactgtgtgcattcatcagcctggtgtggttgagagagcagatagtccatgggggagcaccaatttggttggagcatgctgccccaccgttcaatgctgcggggcatcaccaaaatgaggctccagcaggaggaaatggtgcagaaaatgttgctgctgatcagcctgctaacccaccagctgagaacgcagtggtgggggaaaaccctgatgcccaggatgaccaggcagaagaggaggaggaggacaatgaggaggaagatgacgctggtgtggaggatgcggcagatgctaataacggagcccaggatgacatgaattggaatgctttagaatgggaccgagctgctgaagagcttacatgggaaagaatgctaggacttgatggatcactagtttttctggaacatgtcttctgggtggtatctttaaatacactgttcattcttgtttttgcattttgcccttaccatattggtcatttctcccttgttggtttgggatttgaagaacacgtccaagcatctcattttgaaggcctaatcacaaccatagttgggtatatacttttagcaataacactgataatttgtcatggcttggcaactcttgtgaaatttcatagatctcgtcgcttactgggagtctgctatattgttgttaaggtctctttgttagtggtggtagaaattggagtattccctctcatttgtggttggtggctggatatctgttccttggaaatgtttgatgctactctgaaagatcgagaactgagctttcagtcggctccaggtactaccatgtttctgcattggctagtgggaatggtatatgtcttctactttgcctccttcattctactactgagagaggtacttcgacctggtgtcctgtggtttctaaggaatttgaatgatccagatttcaatccagtacaggaaatgatccatttgccaatatataggcatctccgaagatttattttgtcagtgattgtctttggctccattgtcctcctgatgctttggcttcctatacgtataattaagagtgtgctgcctaattttcttccatacaatgtcatgctctacagtgatgctccagtgagtgaactgtccctcgagctgcttctgcttcaggttgtcttgccagcattactcgaacagggacacacgaggcagtggctgaaggggctggtgcgagcgtggactgtgaccgccggatacttgctggatcttcattcttatttattgggagaccaggaagaaaatgaaaacagtgcaaatcaacaagttaacaataatcagcatgctcgaaataacaacgctattcctgtggtgggagaaggccttcatgcagcccaccaagccatactccagcagggagggcctgttggctttcagccttaccgccgacctttaaattttccactcaggatatttctgttgattgtcttcatgtgtataacattactgattgccagcctcatctgccttactttaccagtatttgctggccgttggttaatgtcgttttggacggggactgccaaaatccatgagctctacacagctgcttgtggtctctatgtttgctggctaaccataagggctgtgacggtgatggtggcatggatgcctcagggacgcagagtgatcttccagaaggttaaagagtggtctctcatgatcatgaagactttgatagttgcggtgctgttggctggagttgtccctctccttctggggctcctgtttgagctggtcattgtggctcccctgagggttcccttggatcagactcctcttttttatccatggcaggactgggcacttggagtcctgcatgccaaaatcattgcagctataacattgatgggtcctcagtggtggttgaaaactgtaattgaacaggtttacgcaaatggcatccggaacattgaccttcactatattgttcgtaaactggcagctcccgtgatctctgtgctgttgctttccctgtgtgtaccttatgtcatagcttctggtgttgttcctttactaggtgttactgcggaaatgcaaaacttagtccatcggcggatttatccatttttactgatggtcgtggtattgatggcaattttgtccttccaagtccgccagtttaagcgcctttatgaacatattaaaaatgacaagtaccttgtgggtcaacgactcgtgaactacgaacggaaatctggcaaacaaggctcatctccaccacctccacagtcatcccaagaataaagtagttgtctcaacaacttgaccttcccctttacatgtccttttttgtggacttctctctttggagatttttcccagtgatctctcagcgttgtttttaagttaaatgtatttgacttgtgttctcagcattcagagagcagcggtgtaagattctgctgttctccctggatcttctgacattactgctgtctgagatttgtatatgtgtaaatacaagttccttgataccctaaaaccttggattaaacagaatgtgcattgtacatctttaaacaaaatgtatattaatttattaaatctagttgtcactttattttggacctgctgtgatctcgacaggaaacgtgccacagagcagtagtgcgcaggcaagacttttcagtgacgccttgtggaacgcagttcatgatgtcctagcagctctcactaagggaactgtacattctttctttcttggctattcagaccttaccaagaacgttaaaggaaacaagtagaaatcagcagtggagtgtctgtggtaagaaaacatgaactttatgcttcactgttagttgtttgtggaagttattttgtataacaccaaagctgttgtacatttcctactgcctgatttttttcatgtgtctgtgtttgtaatattgtatagtatcttgtgctaggtgaggaaattatttttaattttgataatttaatattcctagtgtgatcagcattgggagttgggtttcagtggggcatgtctatacttagagaaaaaaagtccaaatgaagattttcatgagtcagcccccccgcccgcccccaccccacacccacatcctctcttttccacacacaactatctgtttattttttgtagcagtggccgaaagtcctgcaaggtcataaatctttcagagtgacatcaccaactgtactgcatcttactggatttaggacttctgagatgcttgtgaagtatagatgtggttgtggtcttagattgacagcattagagaagactggttagaacatctggtctcgctggttagtgcctcgttggctgaggactaggtgtgcatttctcctagcttttcatcaggaaatcccaaagtttccaaagctttttgtttacagaataaaacttcaaataaaaccaattcattatttgtccagaaggaagcttggctgagctggccttttaacataggaatgtatttcgttggaaacattctgaaaaatctcagagaactgaacccttacaaactttgttttccctcataaccaaagcttcaggttagaagtttagaaaaatagaatggttgggtacatgatctaaatgtttaatgctaaaggtatatcgtaagggtagtgtttgtttttgaacgataatttagaagttctcatagaaagcgtataacataggtcttcagaaactataaaagaattttcatatagtattaaaatccatagactaaaatctgagaattttttaacatatgcaagtcagccaaacataagctaccaaaataaagagcaatgtgttctggctgttttatacttcaacaattttttccctaagtggtaagcaattactttaaaacatatttttaaaaacatcggtatcgggagctgcggtggctccggccggttgtcctggcacacaaggaggcgaggctatgcgttcgaggccaacctaggcaaaattggaaaaaaaaaaaaaaatcagtatcagaaataatgcttgacataggattcagtgttatactctttggcactttagagcagccttttctctcatttgaaataaggtctttgttagccctacctttactcagaaatttagtcctactataaaaaattaggattttaaaatataatgctgcctgtcatagtgtatttctaaattgctctctttgggagaaaaacattattattgaattgagcatataactatataactatataactatatatatatatatatatatatatatatatatatatatatatatatatatatatatatttttttttttttttttttttttttttttttgcccccgagacagagtcttactctgttgcccaggctggagtgcagtggcacgatcctggttcactgcaccctctgcctcccaggttcaagcgattctcctgcctcagcctcccaagtagctggtactacaggtgcgtgccaccacacctggcttattttagttattttagtagagatagggtttcaccatgttggccaggctggtctcggaactcctgactgcaggtgatccgtcctccttgggctcccaaagtgctgggattataggtgtgagcccccacgcctggccaacttgattatattaattttgggttttttgttttgttttgttttgttttttaatttttcaaagaatttgtggatcttggtataaagtcattggaacatatctgttttaacagcaagagatcctaggcagatacttcaaaaggttaaaaattcttaatcctacagaattttaaatgaatcttatcaatgttttgtaaacaaacaatatgaatggccaaaaaattgccctccattttactggcaggtaatttatattgtcttacttaagaattctcccctagtttttcaattgtactttatcgtgttgttttcaatgagataagtattttcatagggaaagcattttccagcataatatttgcttgggtagcatccgggttttagtatttaaccaagagccttttaaatattgaaaacccatagttcagaaaatgttagtattgctgcccttcttcacataaatttttttttaaattatactattattttgcttaattttatattgggttaaaacaaccttcaagaaggttaactaggaaagaagacctttttgttttatttttactatttatatatagaagacaaatcagcatttggtgatagttttacatgaccagttatcaaacggtcatagtatgaagtgtgcagttgttcattattagtaaattatgtttgatttttaaactatttagtactaatagttgagatgaaaactgaagaaaaatgccaatgtgacgtttgtgtatagctagccttaaaaaacttcccatgtttttaggtgacttttttccccctcttagtactctggagaaacaatgaagatgggccatctcaattccagatgtaaacaaaaagtaatttttatttcaacatttaatgtaactgctattattgtggattcttgtcttgtgtattttctttcccttattcaagtaatatagaataactttccttaaaatgatttgatccaagatacgtcatttctgtattggcaaaatgccactattaaagtgtaattcttgaatttaggttgaattgatgactttttaaaacaaaacaaacactggacagttctacattgtattgcgtttgcgaatgtgcgtgaacacacgcacacgtgcaggagaatgtagtgccataagaacactggcgctttttaaaactttccaagctagctacttattttcattttcagggttgagtactctaagctctctacttactgtgagaagttttctacattgtaaaattaaaagattatatttaaaatacttctttaggatgttattcagccatcaaaaaaaaacccaactaaaatgtcttaccagtaaagtattattaaaagcctcatttgtggagattcgctgacttccttggttaagctgtcttgtatttacttaatttgtcccctcaaccaggtgttttcttttcagcctgttactgtcctgtgccatttttaaaaatagccatcatgagtaagggcttctttaaaataatccctgacaatatctttggcattaatacgacataaagacaagacagtatatgtatgttttcattcctgttttgtacgagtgcctcattttgtagtcatgaaagtaacatagtaccttaaataaaaatgtaatgactaatctaaggtctaggaataactattgtagaagctaagtagaacacttagtttggggtgtcaattttctatcaacgcaaacaccttagggtacccagaccaattttatgtatatatattctgaaatgctccctttttaatgtggtcatttgttgccttctaagttgagaggacaaaactgaataaagtgaatgttaattgtaa

SEQ ID NO: 2 - protein, 966 aa, Homo sapiens
MQPLSLSLSPLLPLASSLAPERTHLPGPGSLLLSPPSFPARPREPRGCVTAAPPDKMDTAEEDICRVCRSEGTPEKPLYHPCVCTGSIKFIHQECLVQWLKHSRKEYCELCKHRFAFTPIYSPDMPSRLPIQDIFAGLVTSIGTAIRYWFHYTLVAFAWLGVVPLTACRIYKCLFTGSVSSLLTLPLDMLSTENLLADCLQGCFVVTCTLCAFISLVWLREQIVHGGAPIWLEHAAPPFNAAGHHQNEAPAGGNGAENVAADQPANPPAENAVVGENPDAQDDQAEEEEEDNEEEDDAGVEDAADANNGAQDDMNWNALEWDRAAEELTWERMLGLDGSLVFLEHVFWVVSLNTLFILVFAFCPYHIGHFSLVGLGFEEHVQASHFEGLITTIVGYILLAITLIICHGLATLVKFHRSRRLLGVCYIVVKVSLLVVVEIGVFPLICGWWLDICSLEMFDATLKDRELSFQSAPGTTMFLHWLVGMVYVFYFASFILLLREVLRPGVLWFLRNLNDPDFNPVQEMIHLPIYRHLRRFILSVIVFGSIVLLMLWLPIRIIKSVLPNFLPYNVMLYSDAPVSELSLELLLLQVVLPALLEQGHTRQWLKGLVRAWTVTAGYLLDLHSYLLGDQEENENSANQQVNNNQHARNNNAIPVVGEGLHAAHQAILQQGGPVGFQPYRRPLNFPLRIFLLIVFMCITLLIASLICLTLPVFAGRWLMSFWTGTAKIHELYTAACGLYVCWLTIRAVTVMVAWMPQGRRVIFQKVKEWSLMIMKTLIVAVLLAGVVPLLLGLLFELVIVAPLRVPLDQTPLFYPWQDWALGVLHAKIIAAITLMGPQWWLKTVIEQVYANGIRNIDLHYIVRKLAAPVISVLLLSLCVPYVIASGVVPLLGVTAEMQNLVHRRIYPFLLMVVVLMAILSFQVRQFKRLYEHIKNDKYLVGQRLVNYERKSGKQGSSPPPPQSSQE

SEQ ID NO: 3 - protein, 90 aa, Homo sapiens
MCVNTRTRAGECSAIRTLALFKTFQASYLFSFSGLSTLSSLLTVRSFLHCKIKRLYLKYFFRMLFSHQKKTQLKCLTSKVLLKASFVEIR

SEQ ID NO: 4 - DNA, 6187 bp, Mus musculus
  CDS (156)..(2897): CNGM0002
tgtaagctgggtatggaagcatttggagactgtcacaggagagcactgagttcaaggttaacctgggctacataatgggatcctatcttaaaagaaaaagaaagaaggaaagaaagaacagaaagagagacctctgtaaactggctcatcagtgagtgaagagagactactcaggattgttagaagatatatgtagagtgtgtcggtcagaaggaacacctgagaaacctctttatcatccttgtgtatgtactggcagtattaaatttattcatcaagaatgcttagttcagtggttgaaacatagtcggaaagaatactgtgaattatgcaagcacagatttgctttcacaccaatttattctccagatatgccttcacggctaccaatccaagacatatttgctggactggttacaagtattggcactgcaatacgatactggtttcattacacacttgtggcctttgcatggctgggagttgttcctcttacagcatgccgcatctacaagtgcttgtttactggctccgtgagctcactactgacactgccactagacatgctgtcaacggaaaatttgttggcggattgcttgcagggctgttttgtggtgacgtgcacactttgtgcgttcatcagcctggtgtggttacgagagcagatagtccacgggggagccccaatttggctggagcatgctgctccacccttcaatgctgctggacaccaccaaaatgaggctcctgtaggaggaaatggtgcagaaaaccctgctgctgatcagccagctaacccagcaggtgagaatgcagttctgggtgaaaaccctgatgcccaggacggtcaagcagaagaggaagaggaggacaatgaggaggaagatgatgcgggtgtagaagatgctgctgatgccaacaatggagcccaagacgacatgaactggaatgctttagagtgggaccgggctgcagaagagcttacttgggaaagaatgcttggacttgatggatcactagttttcctggaacacgtgttctgggtggtgtctttaaatacattgttcattcttgtttttgcattttgtccttaccacatcggccatttctcccttgttggcttggggtttgaggagcatgtccaagcatctcattttgaaggtctaatcacaacgattgttggatatatacttttagcaataacactgataatttgtcatgcattggcaactcttgttaaatttcataggtctcgtcgcttgctgggcgtctgctatattgtggttaaggtctctttgttagttgtggtagaaatcggagtgttccctctcatttgtggctggtggctggatatctgttctctggaaatgtttgatgctactctgaaagatcgagagctgagctttcagtcagctccagggaccacgatgtttctgcactggttggtgggaatggtgtatgtgttctactttgcctccttcatcctgttactgagagaggtacttcgacctggtgtcttatggtttctgaggaatttgaacgatccagattttaatccagtacaggaaatgattcacttgcccatttatagacatctccgaagatttattttgtcagtgattgtctttggctccattgtccttctgatgctctggcttcctatacgtataattaagagtctcttgcctaattttcttccttacaatgtcatgctctacagtgatgctccggtgagtgagctgtcccttgagctgctcctactgcaggttgtcttgccagcattgctggaacagggacacacgaggcagtggctgaaggggcttgtgcgtgcatggactgttactgctggatacttgctggaccttcattcctacttactgggagatcaggaagaaaacgagaacagtgcaaatcagcaggtcaacaataaccagcctgcacgaaacaataatgctgttcctgccggggaaggtctgcatgcagcccaccaagccatactccagcagggaggacctgttggctttcagccttaccgccggcctttaaatttcccactcaggatatttctgctgattgtcttcatgtgcataacattgctaattgccagcctcatttgccttactttaccagtatttgctggccgttggttaatgtcattttggacggggactgccaaaatccatgagctgtacacagctgcttgtggtctctatgtgtgttggctaaccataagggctgtgacggtgctggtggcctggatgccccagggacgaagagtgattttccagaaggttaaagagtggtccctcatgataatgaagacgttgatagttgctgtgctgctggctggggtcgtcccactgctcctcgggctcctgtttgagctggttattgttgctcctctgagggtccctcttgatcagactccccttttctacccttggcaggactgggcgctgggagtgctgcatgccaaaattatcgcagctataactcttatgggtcctcagtggtggttgaaaactgtcattgaacaggtttacgcaaatggcattcgtaacatcgaccttcactatatcatccgtaagctggcagccccggtgatctctgtgctcttactttccctgtgtgtaccgtatgtcatagcttctggcgcggttcctctactaggtgttactgcagagatgcagaacttagttcaccggcggatttacccatttttattgatggtcgttgtgctgatgggaatcctgtccttccaggtccgccagtttaagcgcctttatgaacacattaaaaatgacaagtaccttgtggggcagcgcctggtgaactatgagcgcaaatccggcaagcaaggcccgtctacaccgcctccggtgtcgtcccaagaataaagtgcttgtctcagctccttgagcttctccttgccttttcgtgtcctttgttgtggacttctcacttggagattctcctgtgatctcagcgttgtttaagttaaatgtacttgacttgtctcctcagcattcagagagcagcgcatccggcactactgctcctccctgcatctgccgacatcactgctgtcttgagatctgtacatgtgtaaataggagttccttgatgccctaaaaccttggattaaacagaatgtgcattgtacatctttaacaaatgtatattaatttattaaatctagttgtcactttattttggacctgctgtgatctcgacaggaaatgtgccacagagcagtagtgtataggcgagactttgagtgacatcttttggagcacagtgcatggtgttgccagcagttctcaccaggtaactgcatcctgtccttggctggtcagtccttccttaccaggagccgaatgggcagtggagcctgtggtaggggactggacggagtgcctcacagacagtcgtatttggaaattattacatgtaacaccaaagctgttgtacattttctactgcctgattgttttcatgtgtctgtgtttgtaatattgtatagtatattgtgctagatgaggaaattatttttaattttgataatttaatattcctagtgatcagcattgggagttggggtttttgtgcttgttaggggcatgtctatacttagaaagataaactacaaatgaagatttttttattgacctcctccccttccatacagctctttctttttgtcaacagtggccaaaagtgtcatgccggatgagaaatctaaggggatgtcatcaccaacaggaccagtagtgacaggtcaaggccctaggccgtggaatgaggccgactgcatcagggacgatgagtagaacatcctgccgtgctggcagcacattgcagctgaggctcagttaatataccaggaaacccctgttcctaaggcatggtgtttacagtaaaccgcaaataaaaccaagttttatttgtttgaaaggaagcagactgagttggccttcgccataggaaagagttgcattggagaaatgctgtgagattttagagaatttaatactcagaggaagcctggttcccctagcccaagcttcatgtttaaagtttaaaagattaggatggttgtacgtcattcaagtgtttaatggtaaaggtgtattataaggatagtgctagaagctttcatagactgtaaagagttccatggagtattaaacccatagttggaaaggtctgagggcccacccccacctctaaatacaaagccaaacaaaagattaagagcaatgctttctggttttgttcctttcttgattttgtctttttgtcgttcttaaattcactaagtaataagcaaatactttgttttaaagcagcattatatttaaaaaacaaaaacagtcttgccatggatccagtgttaacactgtcattttgtaacagcctttctctcctgtagcaagcctaccttcactctcagactttgtccttccataaagaattagcattttaaaatgcagtgccacctgtcaatgggaatatgtctataaagcctaatcttttgatgctggtatattggacatataacattcaggtttcatttttaatttttaaacaatttatggatctaccacaaaaagctaagcattctcagtcatctggaattgtaagtgagcttactggatgcttttctgagaacaaccacaaacagcgccgattgccaaggtgggccccatctcactggcagggcctgctctcctgtgtgctttgtccagtggctttcaggaagatgatgtcctcgtggggatttattgtgcataacatttgctagggtgacatacaggttttattcaacaatatcataaacatttaaaaccctcaattcagaaggtgttcctatttgctgccggtctttacaggaaatttatagagatgtgcatcattttgaattaggctaacacccagtcttcaagggagtttactaggaaagacctttgtgctttattattgataatttgaagacaaatcaccacttgggattagttttacatgaccagttatcagatggtcacttcgaaatgtatagttgttcattattaacaaattatggtttgatttttaaactacaaagtactaatagtttagatgaaaactgaagaaaatgccaatgtaaaatttgtgtgtagctagccttaatgttccatgtttatagttgtggttccagcccccactcccgttctccgggggtagggtagggagataagacatgtattgaacagtaatttttgttttaacagtcagtatagctgttattactgtgaattctcaagatggtccctcccctcaagggacaatcctcctatgagtttatcagacatgtcgcttctgtgtcgcacaaatgctttattaaagtatattcctaaattcaggttgagttgatgaatttttaagacaaacacaaaatacgcattggacatttgtaccctgttgtgtttgcagacacaggagccagtgccattatgcaagaacttggcacttggcacggtgctccaaattagttccttcctctcctctctggggtggagaatatcaggctagttactgtgatgcctcattttacattgtgatgctcaaagttcttgtttaaaacaatcctctaggatcgtaactgttcagtcattatcaaggggagagatgacatagtaccactgaagtgttactagagacctcatctgtgaagggtctttaaattctttgcttaaattgccctgtagcggggcacttaatttgccctgtcaatcagatgttttgtttcagcttattgctgttgttgtcaaagatttaaaacagctgtcgtgagtaagggcttcttcagatactctccaataatagctttggcattaatataaaaataagacagtatacatgtgtatgtatgtctccattcctgtctatctgtctgagtgcctcattttgtaatcatgacaatcctatagaaccttagatggaaatgagtgactaaggcctaggggtgtctgtttaggagtgaattagaacacttagtgatgtgggaggtcggttttctattacagtaaacacctgtacccagatcaattttatgtgtatatattcaaaaatgctcccttttttatgtggtcatttgttgccttccgagttaagagaacagaattgaataaagtgaatgttagttgta

SEQ ID NO: 5 - protein, 913 aa, Mus musculus
VKRDYSGLLEDICRVCRSEGTPEKPLYHPCVCTGSIKFIHQECLVQWLKHSRKEYCELCKHRFAFTPIYSPDMPSRLPIQDIFAGLVTSIGTAIRYWFHYTLVAFAWLGVVPLTACRIYKCLFTGSVSSLLTLPLDMLSTENLLADCLQGCFVVTCTLCAFISLVWLREQIVHGGAPIWLEHAAPPFNAAGHHQNEAPVGGNGAENPAADQPANPAGENAVLGENPDAQDGQAEEEEEDNEEEDDAGVEDAADANNGAQDDMNWNALEWDRAAEELTWERMLGLDGSLVFLEHVFWVVSLNTLFILVFAFCPYHIGHFSLVGLGFEEHVQASHFEGLITTIVGYILLAITLIICHALATLVKFHRSRRLLGVCYIVVKVSLLVVVEIGVFPLICGWWLDICSLEMFDATLKDRELSFQSAPGTTMFLHWLVGMVYVFYFASFILLLREVLRPGVLWFLRNLNDPDFNPVQEMIHLPIYRHLRRFILSVIVFGSIVLLMLWLPIRIIKSLLPNFLPYNVMLYSDAPVSELSLELLLLQVVLPALLEQGHTRQWLKGLVRAWTVTAGYLLDLHSYLLGDQEENENSANQQVNNNQPARNNNAVPAGEGLHAAHQAILQQGGPVGFQPYRRPLNFPLRIFLLIVFMCITLLIASLICLTLPVFAGRWLMSFWTGTAKIHELYTAACGLYVCWLTIRAVTVLVAWMPQGRRVIFQKVKEWSLMIMKTLIVAVLLAGVVPLLLGLLFELVIVAPLRVPLDQTPLFYPWQDWALGVLHAKIIAAITLMGPQWWLKTVIEQVYANGIRNIDLHYIIRKLAAPVISVLLLSLCVPYVIASGAVPLLGVTAEMQNLVHRRIYPFLLMVVVLMGILSFQVRQFKRLYEHIKNDKYLVGQRLVNYERKSGKQGPSTPPPVSSQE

SEQ ID NO: 6 - DNA, 3905 bp, Drosophila melanogaster
  CDS (299)..(3265)
aatcagcaatttcatggtgtgcacacattggctctcctttttttgagaagtagtcttgttttgttttgtcgtaataatatcccaacttttcggttaaaattcaagtgttacaatcgcattacggtccgtaatcatcgaaaggttaattggcgcacataaaaggcgggtataatcgtcttaaaacccccgtaccctcgggtcaattacggaaattgtccagcgaatttcgcaaatgaccagtggaactttagtaattcagtcaaactgaagacccaggatccgggaaaaagttatcagtatggacgacctgtcgcagggcgacatttgccgcgtgtgcagatgcgaggcacagccggataggcctcttttctacccgtgcatctgcactggatcgatcaagtacattcatcaggactgcctgatgttatggatgcgctactcgcacaaggaatactgcgagctgtgcagctacagcttcagctttcagcccatctatgctccggatatgccgcgagtcctgccgataaaggatgtccttgtgggactgatgtccgccgttttggagggcgctcgctgctggcttcattacaccctggtcggtttggcctggttcggagtggtgccgctgtctgcctacaggacctatcggtacctcttccgggcgagctccttcgatatgattctcacgctgccctttgacatcttctccgttgaaaacctggcatcggatgcctttcggggctgttttgtggtcacatgcacacttttgtccttcataggattggtctggctgcgcgagcagatcctgcatggcggcggacccgattggctggagcgggacgatgcccctctacaggcagcggctgctaatcctgcgcctgatcccgccgccgaggcagctccacagcctcaggacgacaacaacaatgccgacgacaatgaagcgccggctccagcggacaacgatgggcaggatgcccaggcccaggatgccgcaccagcggctgcagctcccgtcgtggacgccgatgccgatgagcagaattggaatcccatggagtgggatcgggctgccgaggagcttacctgggaacgcctgctcggactcgatggctcgctagtttttctggagcacgtcttctggatcatctcactgaacaccatgttcatcttcaccttcgccttttgtccctactgcgtgggcaacttcattctgagctccatggatcttttgcagcccgagaaaccactgctacactttcacggcctgattaccacgctatttggttactgctgcattggattgaccctggtggtgctgcacttcttctccagagttttccgcctgcggagagtctgctggttcattggattgtgctatattgtggtcaaggtgtcgctgctgtcggtgttggagatcggagtactcccgctgatttgcggctggtggctggacatttgttcgttgcccctactagatgccagcctcaaggatcgtaaagagagttttaaggctgctccgggcacctcactttttgtccactggatgtttggcatggtatacgtttactacttcgctgcgtttgtatcgttgttgcgcgaggtgctgcgaccgggagtgctctggatattccgcaatgtcaacgatcctgacttcagtcccatccaggaaatgatccatgtaccaattgtgagacacatccggcggctggtcgcttcggctatgatctttggatttgcagtgctcttgatgctctggctgcccattagaattctgcaggtggtttgtccggacttcttgccatatgcacttagtggcgattcagcggttaacttgcaattgctgctcttacaaatcgttttgccgggcttcttcgagcagacccaaacacgcatctggctaaagggcttgctgcgcatttggtgcacagcggtggcctggttgctgggcatacgaagctacctgctcccagcaccggagccggaaccggaaattcccgctgctggagaagaaggcgtggagaatgccgctggcgaaaatgaaaaccaggaaggtgaggcagcggatgcggcagcccaaccaccagcaccacctcctccaccgccaccgccagtggaaccggcgccggcaccacgaaaccttgccgccgcccatcaggccatcatgcaacgggatgcacccgtgggattccagccgtacgacaagccatcgctgttcgctttacgtttgtgtgccctgttggcactcatgtgtttgtccatcgtttgtgctgccatgttgacgctcacggttccagtctacatcggtcggcggctgatgttgatctggtcctcccagccggatgagaaggcagctggagcggcagccgcagaaggagcgggcgaagtggttgaaaatctagatccaatcgatccggaaggagccagaaagaacgagcgactactgcgctcgcatgagctgtacaccgctgagcttggtggttacctgtgctggattgtctgccgggtagtagctgcggtggtcaccctgctgccacaaggccgcgcggctattgtcgacaagataaagcactgggtccgagtggcattgcaatatgccctgcccgttctaacactcctgggaatcttcgtgctggttccgttgctgattggattgctgatcgagttggcggtggtcattcccctacgcgttccactacgcaagacaccaatccacttcctatgggaggattgggcgctgggagtgctctactgcaagatagccatcgccttgacgatgatgggcccagattggcacttgaagcgtgcattagatcgagcctacatggatggattgcgcgatttcgacttgaagtttgtgatgcgagacttggctgtgccggtcgttactaccattggtctggccctggccataccctatgtcatggcacatatgatgtttccgattttcctagcggacgcctatgctcagcatgtcgtcgcacggctcgtgtatcccgtaagcttcattgtggtgggcagcatatgcatccttttgttccagatcgatcagctgaagaagctctatctgtccatcaaggtggataagtacctggtgggtcagaggctggtcaactatgagcaccgcaagaagcagcaacagcagcaggaggaggagaaggagcaacagcgggcggcccgggaattaagggagaaccaggagagagatcgggagcgagagcgggaacagttggtccaggagctcgagttggctgaacttctgtaacgagatcaggtgcgaagcgtaggtttttcagtttctaatatgtgcgacaggaggaggggaatcaataaattatcagctcatggaggctattaggttctcacgtttaaaattgttaaactctttgaaatatttaagtttttctattgataataatgatggcaatcttttgtttctatcttaagaaattaaagcatgtccaatttctatttaaagatttttatttcaacccgacaaggagaaggacaccagaatgttgaagccgttgaagtattcgtcgtgctttttcgtcgtggagatgcttctgctgctaatatatatcccctgtttagtatctattgctctgcgttacgcaaggatttggatttggatttcgttatagcttagtacctttgttttcccaatcttattatcctttgcaatacgattaatttgttagctgcaattgttgttaaatccttacgctgcacagtttatagatcctaagttgtatgatattttgacttccgccacatccttacaagcacttacagaactaaacgatgggcagcgattaaaccttgtattagagacgtatgcagaaactacatctgagagttgtcaactttaggaactttagagaaaccccaaaaaaaaaaaaaaa

SEQ ID NO: 7 - protein, 988 aa, Drosophila melanogaster
MDDLSQGDICRVCRCEAQPDRPLFYPCICTGSIKYIHQDCLMLWMRYSHKEYCELCSYSFSFQPIYAPDMPRVLPIKDVLVGLMSAVLEGARCWLHYTLVGLAWFGVVPLSAYRTYRYLFRASSFDMILTLPFDIFSVENLASDAFRGCFVVTCTLLSFIGLVWLREQILHGGGPDWLERDDAPLQAAAANPAPDPAAEAAPQPQDDNNNADDNEAPAPADNDGQDAQAQDAAPAAAAPVVDADADEQNWNPMEWDRAAEELTWERLLGLDGSLVFLEHVFWIISLNTMFIFTFAFCPYCVGNFILSSMDLLQPEKPLLHFHGLITTLFGYCCIGLTLVVLHFFSRVFRLRRVCWFIGLCYIVVKVSLLSVLEIGVLPLICGWWLDICSLPLLDASLKDRKESFKAAPGTSLFVHWMFGMVYVYYFAAFVSLLREVLRPGVLWIFRNVNDPDFSPIQEMIHVPIVRHIRRLVASAMIFGFAVLLMLWLPIRILQVVCPDFLPYALSGDSAVNLQLLLLQIVLPGFFEQTQTRIWLKGLLRIWCTAVAWLLGIRSYLLPAPEPEPEIPAAGEEGVENAAGENENQEGEAADAAAQPPAPPPPPPPPVEPAPAPRNLAAAHQAIMQRDAPVGFQPYDKPSLFALRLCALLALMCLSIVCAAMLTLTVPVYIGRRLMLIWSSQPDEKAAGAAAAEGAGEVVENLDPIDPEGARKNERLLRSHELYTAELGGYLCWIVCRVVAAVVTLLPQGRAAIVDKIKHWVRVALQYALPVLTLLGIFVLVPLLIGLLIELAVVIPLRVPLRKTPIHFLWEDWALGVLYCKIAIALTMMGPDWHLKRALDRAYMDGLRDFDLKFVMRDLAVPVVTTIGLALAIPYVMAHMMFPIFLADAYAQHVVARLVYPVSFIVVGSICILLFQIDQLKKLYLSIKVDKYLVGQRLVNYEHRKKQQQQQEEEKEQQRAARELRENQERDREREREQLVQELELAELL

SEQ ID NO: 8 - DNA, 3087 bp, Anopheles gambiae
  CDS (54)..(2765)
ctgcaccgaagccagcccccgttttccgtgttccgaggcgtaccaacagcacgatggagggtgacatttgccgggtgtgccgctgcgaggcccagtccgaccggcccctgttccatccgtgcatctgcaccggcagcatcaagtggatccaccaggactgcctgatgcagtggatgcgctactcgcgcaaggagtactgcgagctgtgcgggcaccgcttctccttcacgccgatctactcgcccgacatgccgcgcgtcctgccgctcaagtacgtcgcaaaggggctgctcagctcggtcggcagggcggtcaagtactggatacactacacgatcgtggccgtcgcctggctcgggatcgtgccgctgaccgcgtaccgcacctaccggttcctcttttccggctcgatcgaaatggtcctctcgctaccgatcgacatgttctcgaccgaaaacataacgatggacgtgtttaggggctgcttcgtcgtcacctgcacactgtttacgtttatcgggctcatttggctgcgggagcagatcatccacggtggcgggccggactggctcgagcgggacgatccgcagccagacccggcacaccagccacaggaagcgaatgaagctcctgttataccaccggaggcggaaccgttgcgccgtcggccagtaccgccccgggcggccaactttggcgaaccgcccgccgacaatgtgccaatcgatccggcggaaccaccgccaccgcaaccggcgctagcggacgaaccgcccgaagcggccatcaacgagccgggcgacggtgaggcagcggccgacgaagccaactggaacccgatggagtgggagcgggccgccgaagagctgacctgggagcggctgctcggcctggacggttcgatggtgtttctcgagcacgtgttttgggtcgtttcgctcaacaccgcgttcatcgtgctgttcgcgttctgcccgtactcgatcggcaactttctgatcacgtttctgggcatcaatgcgcccggcaagcagctgacgtacttccacgggctgctcaccaccatgctcgggtactgcgtgatcggcatcacgctggtgaagctgcacgcgatcgctcgctttctgcgctggcgtcggcagcggcgcatcctcggactgtgctacatcgtggtgaaggtgtcgctcctgtccgtggtcgaaatcggcgtgctgccgctcgtctgtggctggtggctcgatatctgctcgctgcccatgtttgacgccacgctgaaggaccgcaaggcgagctttaaggtggcgcccggcacgtccctgttcatccactggatgtttggcatggtgtacgtgtactactttgccacgttcatcgtgctgctgcgggaggtgctccggcccggtgtgctgtggtttttgcgcaacttaaacgatcccgactttagcccgatccaggaaatgattcacctttcgattctgcgccacacgcgccgcctgatatcgtcggccaccatcttcgggtcggcggtgctgctgatgctttgggcaccgatccaaatcctcaaaaccttctggcccacattcctgccctacacactgtccggtgattcggaggtgaacgagctcagcttgcagttgctgttgctacagttcgtgctacccagcttcttcgaacagtcgcacacacggatttggttgaagggtttggtgcgcatctggtgcaacgtggtgtcgcgcatgctcggcatcaagagctatctgctcggggcggacccgcaaccgaacgaggacgatgctgcaccgccaccccggcagcagccggacgcaggtgctgggctggccgccgcccatcaagccatcatgcagcgggacgtaccggtcggctttcagccctacgaacggcccaagttcttcgccgttcgtctgatcggtttgctggtgctgatgtgcattagcttggcgatcggttcgctggccatactgaccatcccggtctggatcgggcgctacggtatggcgctggggtcgataggttcgtccaacattgcgcccccgccgagcgcggtcagcagcacgggcacggaaacgccggcccggccacacgagctgtacaccgccgccatcgggatgtatctgtgctggctaatttcgcgtggcatcgcgctggccgtgaatctggtgccgcagggtcgggctgccgtcattgcacgcatcaagcactggatttcgatcggcacgtcgtacgcgctggccatgttcgtgttcgtactgatgctcggcgtgattccgctcatgttcgggctgctgctggagctggtggtcgtgataccgttgcgcgtgccgcccgatcaaacgccggtcctgttcctctggcaggactgggcgctgggcgtgctgtacacgaaaattgcctgcgccctcacgctgatgggaccggactgggcgctgaagcgcgcgatcgagcaggcgtaccgggacgggctgcgcgacatcaacctgaactttatcgtgcgcgagctcggcatcccggtcattacctgcttcgggctggcgctggccgtcccgtacgtgatcgcgcactcgattctgccggtgttcttcccgaacccgctcacccgcacgctcatctcgcgccagatttatcccttcttcctgctcattgcgttcgtgatcggcatagtggtactgcaggtgcgccagttccggaagctgtacgtagcgatcaagaacgacaagtatttggtcggccagcggttagtgaactaacgatcatcagcgtaagaagaggcgcccgtcggtggtggcgactgaagcgcaagaggctaccggggcggcagcgtcggcggcggcggcggctgcaggggacaccggaggtgatgcggcccggtcggccagcgatgccgctaaccccccgtcctcggcgatggaaccaaacaacgatggtagtacggctgaagcggcggcggcggcagctagcgacgaacccggcagcaccagtggccggatccgtgtcgcgatggaagctgtacagtagaaggagtgaacaatcatgcagtgtgtgtgtgtatgtgtgtgtgtatggggcgtg

SEQ ID NO: 9 - protein, 903 aa, Anopheles gambiae
MEGDICRVCRCEAQSDRPLFHPCICTGSIKWIHQDCLMQWMRYSRKEYCELCGHRFSFTPIYSPDMPRVLPLKYVAKGLLSSVGRAVKYWIHYTIVAVAWLGIVPLTAYRTYRFLFSGSIEMVLSLPIDMFSTENITMDVFRGCFVVTCTLFTFIGLIWLREQIIHGGGPDWLERDDPQPDPAHQPQEANEAPVIPPEAEPLRRRPVPPRAANFGEPPADNVPIDPAEPPPPQPALADEPPEAAINEPGDGEAAADEANWNPMEWERAAEELTWERLLGLDGSMVFLEHVFWVVSLNTAFIVLFAFCPYSIGNFLITFLGINAPGKQLTYFHGLLTTMLGYCVIGITLVKLHAIARFLRWRRQRRILGLCYIVVKVSLLSVVEIGVLPLVCGWWLDICSLPMFDATLKDRKASFKVAPGTSLFIHWMFGMVYVYYFATFIVLLREVLRPGVLWFLRNLNDPDFSPIQEMIHLSILRHTRRLISSATIFGSAVLLMLWAPIQILKTFWPTFLPYTLSGDSEVNELSLQLLLLQFVLPSFFEQSHTRIWLKGLVRIWCNVVSRMLGIKSYLLGADPQPNEDDAAPPPRQQPDAGAGLAAAHQAIMQRDVPVGFQPYERPKFFAVRLIGLLVLMCISLAIGSLAILTIPVWIGRYGMALGSIGSSNIAPPPSAVSSTGTETPARPHELYTAAIGMYLCWLISRGIALAVNLVPQGRAAVIARIKHWISIGTSYALAMFVFVLMLGVIPLMFGLLLELVVVIPLRVPPDQTPVLFLWQDWALGVLYTKIACALTLMGPDWALKRAIEQAYRDGLRDINLNFIVRELGIPVITCFGLALAVPYVIAHSILPVFFPNPLTRTLISRQIYPFFLLIAFVIGIVVLQVRQFRKLYVAIKNDKYLVGQRLVN

SEQ ID NO: 10 - DNA, 30 bp, Homo sapiens
catatgtagagtgtgtcggtcagaaggaac

SEQ ID NO: 11 - DNA, 20 bp, Homo sapiens
gtggagatgagccttgtttg

SEQ ID NO: 12 - DNA, 25 bp, Homo sapiens
gatgctaataacggagcccaggatg

SEQ ID NO: 13 - DNA, 25 bp, Homo sapiens
ttctcccaccacaggaatagcgttg

SEQ ID NO: 14 - DNA, 25 bp, Homo sapiens
gatgctaataacggagcccaggatg

SEQ ID NO: 15 - DNA, 20 bp, Homo sapiens
tgcaggactccaagtgccca

SEQ ID NO: 16 - DNA, 20 bp, Homo sapiens
acacgtgcaggagaatgtag

SEQ ID NO: 17 - DNA, 20 bp, Homo sapiens
ggtaagacattttagttggg

SEQ ID NO: 18 - DNA, 30 bp, Homo sapiens
tcattttcagggttgagtactctaagctct

SEQ ID NO: 19 - DNA, 25 bp, Homo sapiens
ttttaaaactttccaagctagctac

SEQ ID NO: 20 - DNA, 30 bp, Homo sapiens
tttaattttacaatgtagaaaacttctcac

(19) United States
(12) Patent Application Publication
(10) Pub. No.: US 2005/0136451 A1
(43) Pub. Date: Jun. 23, 2005

(54) Hypoxia responsive human CNGH 0002 genes and polypeptides

(76) Inventors: Li Yan, Wayne, PA (US); Chris Huang, Paoli, PA (US); Prabakaran Kesavan, Blackwood, NJ (US); Katherine Li, Wallingford, PA (US); Marian Nakada, Malvern, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/957,503

(22) Filed: Oct. 1, 2004

Related U.S. Application Data
(60) Provisional application No. 60/508,706, filed on Oct. 6, 2003.

(51) Int. Cl. C12Q 1/68; C07H 21/04; C12N 9/02

(52) U.S. Cl. 435/6; 435/69.1; 435/189; 435/320.1; 435/325; 530/350; 536/23.2

(57) ABSTRACT

The invention provides isolated nucleic acids encoding a variety of nucleic acids and proteins having diagnostic, preventive, therapeutic, and other uses. These nucleic acids and proteins are useful for diagnosis, prevention, and therapy of a number of human and other animal disorders. The invention also provides antisense nucleic acids molecules, expression vectors containing the nucleic acids molecules of the invention, host cells into which the expression vectors have been introduced, and non-human transgenic animals in which a nucleic acids molecule of the invention has been introduced or disrupted. The invention still further provides isolated polypeptides, fusion polypeptides, antigenic peptides and antibodies. Diagnostic, screening, and therapeutic methods utilizing compositions of the invention are also provided. The nucleic acids and polypeptides of the present invention are useful as modulating agents in regulating a variety of cellular processes.

### CROSS REFERENCE TO RELATED APPLICATION

This application is a non-provisional application filed under 37 CFR 1.53(b)(1), claiming priority under 35 USC 119(e) to provisional application No. 60/508706 filed Oct. 6, 2003, the contents of which are incorporated herein by reference.

### FIELD OF THE INVENTION

The invention relates to newly identified hypoxia responsive genes and fragments thereof, the polypeptides encoded by these genes, uses of such genes and polypeptides encoded by such genes and their production.

### BACKGROUND OF THE INVENTION

With the recent completion of the sequencing and initial annotation of the human genome, and through the techniques of molecular biology, transgenic and null mutant or “knockout” animal production, computational biology, pharmacogenomics, and the like, the role and importance of individual genes and proteins in development and in normal and disease states is within the grasp of understanding.

With the tools enabling detection of the levels of gene expression and protein production in response to stimuli, natural or adverse, the practitioner is able to identify biological targets that are either representative of or critical to the change in physiological state of a cell, tissue or organism. From such knowledge it is further possible to design molecules capable of identifying, quantitating, and modifying the levels of such target molecules. These newly designed molecules are the precursors of potential diagnostic, therapeutic, or protective agents.

The present invention provides sequence information for polynucleotides derived from a human gene and for a protein encoded thereby, and thus enables the practitioner to assess, predict, and affect the physiological state of various human tissues.

One of the most critical and earliest hallmarks during solid tumor disease progress is the phenotypic switch from avascular tumor nodules to vascular tumors (Folkman, (1995), Nat Med 1: 27-31). The acquisition of vascularature, also known as tumor angiogenesis, confers the ability to expand and develop both locally and metastatically on the tumor. Hypoxia, the inadequate supply of oxygen (O2), represents the most important event triggering tumor angiogenic switch, or the conversion from the avascular neoplasm to its malignant, fast growing counterpart (Harris, (2002), Nat Rev Cancer 2: 38-47). In a solid tumor, hypoxia develops where the distance to a neighboring blood vessel is beyond the diffusion capacity of oxygen, typically in the range of 100-150 mm (Thomlinson and Gray, (1955), Br. J. Cancer 9: 539-549). In addition, hypoxia could also develop in areas within a tumor with compromised blood flow due to aberrant vasculature formation and high interstitial pressure (Brown and Giaccia, (1998), Cancer Res 58: 1408-16). Sustained hypoxia in a growing tumor may be responsible for cellular changes that lead to more clinically aggressive phenotypes. Recent laboratory studies suggest that hypoxia contributes to the selection for cells with diminished apoptotic potential, increased local invasive growth, perifocal, regional and distant tumor cell spreading capacity, and resistance to radiation and other treatments (Hockel and Vaupel, (2001), J Natl Cancer Inst 93: 266-76). In the mean time, clinical studies demonstrated that intra-tumor hypoxic status could serve as a prognostic marker associated with a lower probability of disease-free survival and increased incidence of distant metastases (Brizel, et al., (1996), Cancer Res 56: 941-3, Brizel, et al., (1997), Int J Radiat Oncol Biol Phys 38: 285-9, Nordsmark and Overgaard, (2000), Radiother Oncol 57: 39-43).

In short, hypoxia alters fundamental and physiologically important pathways resulting in more aggressive tumor behaviors. Understanding the molecular mechanisms underlying the tumor hypoxia and the subsequent re-oxygenation provide insights into tumor progression, potentially tumor angiogenic switch and invasion.

In addition to cancer, hypoxia has also been indicated to be associated with pathological changes in other chronic disease conditions, such as cardiovascular diseases (Freedman and Isner, (2001), J Mol Cell Cardiol 33: 379-93), diabetic retinopathy (Ciulla, et al., (2002), Acta Ophthalmol Scand 80: 468-77), Alzheimer's disease (Bazan, et al., (2002), Mol Neurobiol 26: 283-98) and pulmonary diseases (Similowski, (2001), Rev Prat 51: 1066-71). Hypoxia also plays a role in neonatal stress and pregnancy related diseases. Therefore, the identification and characterization of novel hypoxia-response genes could also help to elucidate the crucial roles of these genes and their potential as therapeutic targets in these diseases.

A number of hypoxia responsive genes have been identified in the art. See e.g. WO99/48916. Differential-display polymerase chain reaction (PCR) has been used to identify genes induced by hypoxia (O'Rourke et al (1996) Eur. J. Biochem 241:403-410).

### SUMMARY OF THE INVENTION

The present invention is based, at least in part, on the discovery of a human gene whose expression is induced under hypoxic conditions. The invention thus relates to this gene, herein designated CNGH0002, and coding sequences contained therein for expression of polypeptides of the same designation. Proteins, fragments thereof, derivatives thereof, and variants thereof are collectively referred to herein as the polypeptides of the invention or the proteins of the invention. Nucleic acid molecules encoding polypeptides of the invention are collectively referred to as nucleic acids of the invention. The invention disclosed is based on the discovery of a novel gene through data analysis of microarray gene expression profiling of samples obtained in vitro under conditions that recapitulate in vivo changes during tumor progression, specifically during tumor angiogenic switch. Extensive gene profiling analysis of a series of experimental settings was carried out to identify genes with expression levels that are differentially regulated and potentially may be responsible for changes in cellular behaviors during early tumor progression. Further functional analysis of these genes in combination with an assessment of their expression in clinical tumor samples provided insights into novel oncology indications for known genes, and resulted in discovery of novel genes that are critical for tumor progression.

The nucleic acids and polypeptides of the present invention are useful as modulating agents in regulating a variety of cellular processes. Accordingly, in one aspect, the present invention provides isolated nucleic acid molecules encoding a polypeptide of the invention or a biologically active portion thereof.

Thus, one aspect of the invention provides the isolated nucleic acid sequence of the novel human gene CNGH0002 as set forth in SEQ ID NO: 1.

Polynucleotides with sequences complementary to SEQ. ID. No. 1, fragments of SEQ. ID. No. 1 which are at least fifteen nucleotides in length, and sequences that hybridize to SEQ. ID No. 1 are also contemplated by the present invention.

The present invention further provides nucleic acid molecules which resemble a double stranded segment of the gene sequence and its complement and can function in an inhibitory manner to the production of the polypeptides of the invention, such as siRNA molecules or antisense molecules, small hairpin RNA (shRNA), or ribozymes. The present invention also provides nucleic acid molecules which are suitable as primers or hybridization probes for the detection of nucleic acids encoding a polypeptide of the invention.

The invention includes nucleic acid molecules which encode naturally occurring allelic variants of a polypeptide having an amino acid sequence comprising any of SEQ ID NOs: 2 or 3, wherein the nucleic acid molecule hybridizes under stringent conditions with a nucleic acid molecule having the nucleic acid sequence of either of SEQ ID NO: 1, or a complement thereof.

The invention includes nucleic acid molecules which encode a homolog polypeptide of CNGH0002 found in a non-human animal, wherein the nucleic acid molecule hybridizes under stringent conditions with a nucleic acid molecule having the nucleic acid sequence of SEQ ID NOs: 4 and 9 and 11, or a complement thereof. Representative homologs identified in other than humans indicate the conserved nature of the polypeptides of the invention and its functional importance. A mouse homolog gene, CNMG002 (SEQ ID NO: 4) coding for the predicted mouse homolog protein CNMG002 (SEQ ID NO: 5); a Drosophila homolog gene (SEQ ID NO: 6) coding for the predicted Drosophila homolog protein (SEQ ID NO: 7) and an Anopheles homolog gene (SEQ ID NO: 8) coding for the predicted Anopheles (SEQ ID NO: 9) are also disclosed in the present invention.

In other embodiments, the nucleic acid molecules are at least 15 and up to 7082 nucleotides in length and hybridize under stringent conditions to a nucleic acid molecule having the nucleotide sequence of either of SEQ ID NOs: 1 or 4 or 9 or 11, or a complement thereof.

Another aspect of the invention provides vectors, e.g., recombinant expression vectors, comprising a nucleic acid molecule of the invention. In another embodiment, the invention provides isolated host cells, e.g., mammalian and non-mammalian cells, containing such a vector or a nucleic acid of the invention. The invention also provides methods for producing a polypeptide of the invention by culturing, in a suitable medium, a host cell of the invention containing a recombinant expression vector encoding a polypeptide of the invention such that the polypeptide of the invention is produced.

Another aspect of this invention features isolated or recombinant proteins and polypeptides of the invention. Preferred proteins and polypeptides possess at least one biological activity possessed by the corresponding naturally-occurring human polypeptide. Also within the invention are isolated polypeptides or proteins having an amino acid sequence that is at least about 50%, preferably 60%, 75%, 90%, 95%, or 98% identical to any of SEQ ID NOs: 2, 3, 5, 10, or 12.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common domain and/or common functional activity.

In one embodiment, the isolated polypeptide of the invention represents a receptor protein containing an extracellular domain, a transmembrane domain, and an intracellular domain. In another embodiment the polypeptide of the invention contains a truncated or domain deleted extracellular portion. In another embodiment, the isolated polypeptide of the invention lacks a cytoplasmic domain or a portion thereof. In another embodiment, the polypeptide lacks both a transmembrane domain and a cytoplasmic domain and is soluble under physiological conditions.

The nucleic acids coding for the polypeptides of the present invention, or a fragment thereof, can be operably linked to a nucleic acid for a heterologous amino acid sequence to form a fusion protein. A further embodiment is a “mimetibody” which comprises at least a fragment of CNGH0002 fused to at least one immunoglobulin constant region including a mimetibody wherein the immunoglobulin constant region is the Fc portion of human IgG1.

The invention further features antibody substances that specifically bind a polypeptide of the invention such as monoclonal or polyclonal antibodies, antibody fragments, single-chain antibodies, and the like. In addition, the polypeptides of the invention or biologically active portions thereof can be incorporated into pharmaceutical compositions, which optionally include pharmaceutically acceptable carriers. These antibody substances can be made, for example, by providing the polypeptides of the invention to an immunocompetent vertebrate and thereafter obtaining serum or cells containing the antibody or containing the nucleic acid sequences capable or directing expression of the antibody. Alternatively, the antibody may be derived from a library containing a repertoire of immunoglobulin domains by selecting binding partners to at least a fragment of CNGH0002.

In another aspect, the present invention provides methods for detecting the presence of the activity or expression of a polypeptide of the invention in a biological sample by contacting the biological sample with an agent or agents capable of detecting the presence or the activity of CNGH0002 in the biological sample.

In another aspect, the invention provides methods for modulating activity of a polypeptide of the invention comprising contacting a cell with an agent that modulates (inhibits or enhances) the activity or expression of a polypeptide of the invention such that activity or expression in the cell is modulated. In a preferred embodiment, the agent is an antibody that specifically binds to a polypeptide of the invention. In other embodiments, the modulator is a peptide, peptidomimetic, or other small molecule.

The present invention also provides methods of treating a subject having a disorder wherein the disorder can be ameliorated by modulating the amount or activity of the polypeptide of the invention. The present invention also provides methods of treating a subject having a disorder characterized by aberrant activity of a polypeptide of the invention or aberrant expression of a nucleic acid of the invention by administering an agent which is a modulator of the activity of a polypeptide of the invention or a modulator of the expression of a nucleic acid of the invention to the subject. In one embodiment, the modulator is a protein of the invention. In another embodiment, the modulator is a nucleic acid of the invention.

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of a polypeptide of the invention wherein a wild-type form of the gene encodes a polypeptide having the activity of the polypeptide of the invention.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates expression of a polypeptide or nucleic acid of the invention by measuring expression of the polypeptide or nucleic acid in the presence and absence of the compound.

Other features and advantages of the invention will be apparent from the following detailed description and claims.

### BRIEF DESCRIPTION OF THE DRAWINGS

FIG. 1 is a diagrams representing the overall the gene composition of the human gene CNGH0002 (A) and the mouse homolog CNGM002 (B).

FIG. 2 is a schematic showing the relative size and relationship of the known mRNAs in GenBank database against CNGH0002.

FIG. 3 is a diagram showing the predicted protein domain composition of human gene CNGH0002.1 (A), the mouse homolog CNGM002 (B) and the human protein CNGH0002.2 (C).

FIG. 4 is a schematic showing the relative hydrophobicity along the amino acid sequence coded for by CNGH0002 for A) protein CNGH0002.1 and B) CNGH0002.2.

FIG. 5 is a photograph of an agarose gel separation of PCR products of the CNGH0002 gene ORF. The figure on the left panel depicts the cloning of shorter and large version of the ORF. In the right panel, PCR products were purified from the gel and reamplified. Lane 1 depicts the results from cloning of shorter fragment from colon cancer tissue. Lane 2 depicts the results from cloning of the shorter fragment from mammary carcinoma. Lane 3 depicts the results from cloning of large fragment from colon cancer tissue. No band was seen from colon cancer cDNA. Lane 4 depicts the cloning of shorter fragment from mammary carcinoma. A very faint band was visible in lane 4, marked by an arrow. Lane 2′ is the results from reamplification of the shorter fragment from mammary carcinoma. Lane 4′ is reamplification of large fragment from mammary carcinoma. HMr=high range DNA markers LMr=low range DNA markers

### DETAILED DESCRIPTION OF THE INVENTION

### Definitions

The following definitions are set forth to illustrate and define the meaning and scope of various terms used to describe the invention herein.

An “activity”, a biological activity, and a functional activity of a polypeptide of the invention refers to an activity exerted by a protein or polypeptide of the invention in response to its specific interaction with another protein or molecule as determined in vivo, or in vitro, according to standard techniques. Such activities can be a direct activity, such as an association with or an enzymatic activity on a second protein or an indirect activity, such as a cellular processes mediated by interaction of the protein with a second protein or a series of interactions as in intracellular signally or the coagulation cascade.

An “antibody” includes any polypeptide or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion, fragment or variant thereof. The term “antibody” is further intended to encompass antibodies, digestion fragments, specified portions and variants thereof, including antibody mimetics or comprising portions of antibodies that mimic the structure and/or function of an anitbody or specified fragment or portion thereof, including single chain antibodies and fragments thereof. For example, antibody fragments include, but are not limited to Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, et al., eds., Current Protocols in Immunology, John Wiley & Sons, Inc., NY (1994-2001); Colligan et al., Current Protocols in Polypeptide Science, John Wiley & Sons, NY, N.Y., (1997-2001)).

“Chimeric” or “fusion” molecules are nucleic acids or polypeptides which are created by combining one or more of nucleotide sequences of this invention (or their parts) with additional nucleic acid sequence(s). Such combined sequences may be introduced into an appropriate vector and expressed to give rise to a chimeric or fusion polypeptide.

“Complement of” or “complementary to” a nucleic acid sequence of the invention refers to a polynucleotide molecule having a complementary base sequence and reverse orientation as compared to a first polynucleotide.

“Hypoxia” (or “hypoxic”) means, for the purposes herein, an environment of reduced oxygen tension. Such oxygen tension is usually less than about 5%. The term “hypoxia induced” or “hypoxia-responsive” when referring to a gene means the gene is expressed at a higher level when the host cell is exposed to hypoxic conditions. A “hypoxia related condition” is a condition in an animal where hypoxia or altered levels of expression of hypoxia-responsive genes in a tissue of an animal is involved. The hypoxia or altered expression of hypoxia responsive genes may either be a symptom or play a role in the cause, development, progression, amelioration or cure of the condition. A hypoxia related condition may be a disease or pathological condition such as, but not limited to, cancer, ischemia, reperfusion, retinopathy, neonatal distress, preeclampsia, cardiac arrest, stroke and wound healing.

A “polypeptide” is a polymer of amino acid residues joined by peptide bonds, and a peptide generally refers to amino acid polymers of 12 or less residues. Peptide bonds can be produced naturally as directed by the nucleic acid template or synthetically by methods well known in the art.

A “protein” is a macromolecule comprising one or more polypeptide chains. A protein may further comprise substituent groups attached to the side groups of the amino acids not involved in formation of the peptide bonds. Typically, proteins formed by eukaryotic cell expression also contain carbohydrates. Proteins are defined herein in terms of their amino acid sequence or backbone and substituents are not specified whether known or not.

“Nucleic acids” are polymers of nucleotides, wherein a nucleotide comprises a base linked to a sugar which sugars are in turn linked one to another by an interceding at least bivalent molecule such as phosphoric acid. In naturally occurring nucleic acids the sugar is either 2′-deoxyribose (DNA) or ribose (RNA). Unnatural poly- or oliogonucleotides contain modified bases, sugars or linking molecules but are generally understood to mimic the complementary nature of the naturally occurring nucleic acids they are designed after. An example of an unnatural oligonucleotide is an antisense molecule composition which has a phosphorothiorate backbone. An oligonucleotide generally refers to nucleic acids having less than 30 nucleotides.

The term “receptor” denotes a molecule having biological activity resulting from interaction with a specific ligand or binding partner. Cell membrane bound receptors are characterized by an extracellular ligand-binding domain, one or more membrane spanning or transmembrane domains, and an intracellular effector domain that is typically involved in signal transduction. Ligand binding to cell membrane receptors causes changes in the extracellular domain that are communicated across the cell membrane, direct or indirect interaction with one or more intracellular proteins and alters cellular properties such as enzyme activity, cell shape, or gene expression profile. Receptors may also be untethered to the cell surface and may be cytosolic, nuclear, or released from the cell altogether. Non-cell associated receptors are termed soluble receptors.

### Characteristics of CNGH0002

This invention discloses the molecular characterization of a novel hypoxia-response gene CNGH0002 (Centocor Novel Gene Human 0002) using microarray gene expression profiling. The invention sets forth sequences encoding CNGH0002 proteins, and presents evidence for the function of said gene during diseases which underlie hypoxic conditions. Said sequences include nucleic acid sequences of full-length cDNA, open reading frame (ORF)s, probes and primers for detecting the gene using Northern blot analysis or polymerase chain reaction (PCR), or for modulating gene expression using antisense, small inhibitory RNA (siRNA), small hairpin RNA (shRNA), ribozymes, and vectors containing the sequences and the polypeptides encoded by them.

Compositions and methods for the therapy and diagnosis of cancer, ischemia-reperfusion injury, retinopathy, neonatal distress, preeclampsia, cardiac arrest, stroke, wound healing, other cardiovascular diseases, Alzheimer's disease and pulmonary diseases are disclosed.

Among novel hypoxia-response genes identified in this series of studies was an EST AL080234. This EST is mapped to human chromosome 5q15.2, between Gene ASP (GenBank accession NM_{—}031916) and predicted gene LOC134147 (GenBank accession NM_{—}138809). After careful analysis of this region, we assembled a previously undisclosed gene named CNGH0002, which spans BAC contigs AC012640.11 and AC092336.2, from nucleotides 10521547 to 10605820.

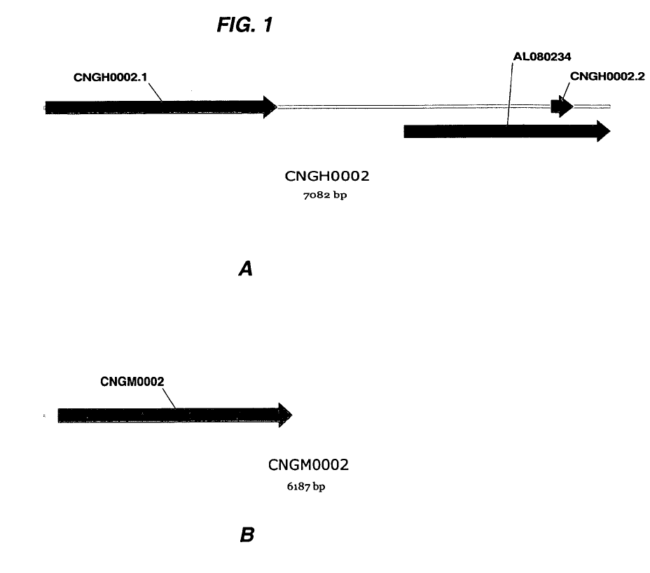

The full length of the cDNA encoding human CNGH0002 protein (FIG. 1A; SEQ ID NO: 1) is 7082 nucleotide residues. Modeled by several gene prediction algorithms, such as GeneScan, Fgenesh, and Acembly program (http://www.acedb.org/Cornell/acembly/) against 350 overlapping ESTs and partial mRNA, a long transcript of 7082 nucleic acids that potentially encodes two novel genes, namely CNGH0002.1 (CNGH stands for Centocor Novel Gene Human) and CNGH0002.2 was predicted.

CNGH0002.1, coded by nucleotides 16-2916, is predicted to consist of 26 exons (SEQ ID NO: 1) and encode a protein of 966 amino acid residues (SEQ ID 2). CNGH0002.2, coded by nucleotide 6344-6616 of SEQ ID NO: 1, is predicted to encode a 90 amino acid protein (SEQ ID NO: 3). A previously known EST, AL080234, which is a component of the microarray “gene chip” used to discover CNGH0002 as described in experiments herein, maps to nucleotide 4490-7082 of CNGH0002. The overall gene composition, together with its two predicted transcripts, is shown in FIG. 1.

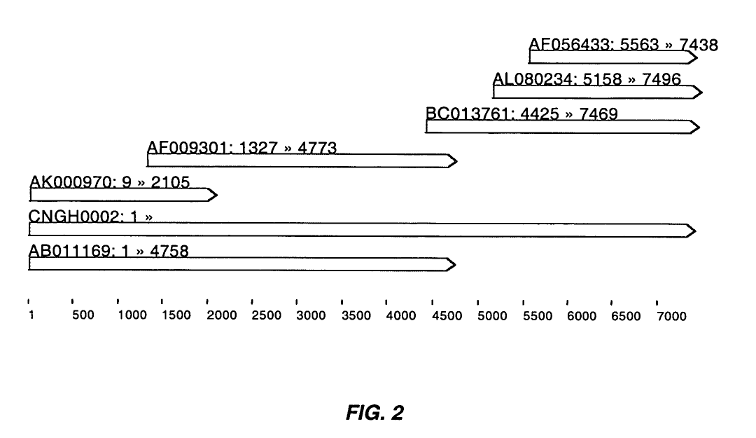

Several previously known mRNA species were identified from the public GenBank database to contain sequences partially overlapping with CNGH0002 transcript. They include AK000970, BC013761, AB011169 (Homo sapiens mRNA for KIM0597 protein), AF009301 (Homo sapiens TEB4 protein mRNA), AL080234 and AF056433. However, none of these mRNA species covers the entire length of CNGH0002 transcript, rendering it a novel gene transcript (FIG. 2).

Based on nucleic acid sequence homology search of CNGH0002, a mouse homologue gene was constructed, Centocor Novel Gene Mouse 0002 (CNGM0002) of 6187 nucleotides (SEQ ID NO: 4). CNGM0002 maps to mouse chromosome 15 and is predicted to have 24 exons that encode a 913 amino acid mouse protein CNGM0002 (FIG. 1 and SEQ ID NO: 5). The protein-coding region of CNGH0002.1 and CNGM0002 are 90% identical.

### Predicted Protein Sequence of CNGH0002.1

By applying domain prediction algorithms such as HMMER, TMHMM, we predict that both CNGH0002.1 and CNGM0002 encode a transmembrane protein with 14 transmembrane domains (TM) and an N-terminal C3HC4 type of Zinc-binding Ring-finger (Zf-C3HC4). In fact the domain structure of these two proteins are nearly identical, since they share 96.8% sequence identity at the protein level (Table 2). The location of each domain in CNGH0002.1 and CNGM0002 is listed in Table 1.

TABLE 1
Domain structure of CNGH0002.1 and CNGM0002 Proteins

| | CNGH0002.1 | CNGH0002.1 | CNGM0002 | CNGM0002 |
|---|---|---|---|---|
| Type | Start | End | Start | End |
| Zf-C3HC4 | 65 | 111 | 13 | 59 |
| TM 1 | 148 | 170 | 96 | 118 |
| TM 2 | 197 | 219 | 145 | 167 |
| TM 3 | 346 | 368 | 294 | 316 |
| TM 4 | 388 | 410 | 336 | 358 |
| TM 5 | 423 | 445 | 371 | 393 |
| TM 6 | 476 | 498 | 424 | 446 |
| TM 7 | 536 | 558 | 484 | 506 |
| TM 8 | 578 | 596 | 526 | 544 |
| TM 9 | 691 | 713 | 637 | 659 |
| TM 10 | 728 | 750 | 679 | 701 |
| TM 11 | 777 | 799 | 724 | 746 |
| TM 12 | 819 | 841 | 766 | 788 |
| TM 13 | 869 | 891 | 809 | 831 |
| TM 14 | 906 | 923 | 851 | 870 |

In addition, we have found CNGH0002.1 homologues in Drosophila protein (SEQ ID NO: 7) coded for by SEQ ID NO: 6 and Anopheles gambiae PEST homolog protein (SEQ ID NO: 9) coded for by SEQ ID NO: 8. These proteins are 58% and 46% identical to CNGH0002.1, respectively (Table 2) and share a domain structure similar to CNGH0002.1 and CNGM0002: a C3HC4 Zinc-binding Ring-finger at the N-terminus followed by 14 transmembrane domains.

TABLE 2
Similarity among CNGH0002.1 and its homologues

| | | | CNGH0002 | CNGH0002 |
|---|---|---|---|---|
| | CNGH0002 | CNGM0002 | PEST | drosophila |
| CNGH0002 | 100 | 97 | 59 | 46 |
| CNGM0002 | | 100 | 58 | 46 |
| CNGH0002_{—} | | | 100 | 53 |
| PEST | | | | |
| CNGH0002_{—} | | | | 100 |
| drosophila | | | | |

The RING-finger is a specialized type of Zn-finger of 40 to 60 residues that binds two atoms of zinc, and is probably involved in mediating protein-protein interactions and in some cases the formation of multi-protein complexes. It has been implicated in a range of diverse biological processes.

The N-terminal portion of CNGH0002.1 and CNGM0002, from the second transmembrane domain to the sixth, shows a distant sequence similarity to the transmembrane region of Frizzled/Smoothened protein family. Proteins in this family are known receptors for the Wnt signaling and hedgehog signaling molecules, respectively (Chen and Struhl, (1998), Development 125: 4943-8, Bhanot, et al., (1996), Nature 382: 225-30). The last 6 transmembrane domains at the C-terminal half the CNGH0002.1 and CNGM0002 bear weak similarity with the transmembrane region of competence proteins. Members of this family are integral membrane proteins with six predicted transmembrane helices. Some members of this family have been shown to be essential for bacterial competence in transporting extracellular DNA across the cell membrane (Hahn, et al., (1993), Mol Microbiol 10: 99-111).

Based on the above prediction of protein structure, CNGH0002.1 and CNGM0002 are membrane proteins involved in mediating protein interactions, that is, the proteins encoded by the genes of the invention function as receptors for unknown ligands. In addition, structural features indicate a role in DNA transport.

### Predicted Protein Sequence of CNGH0002.2

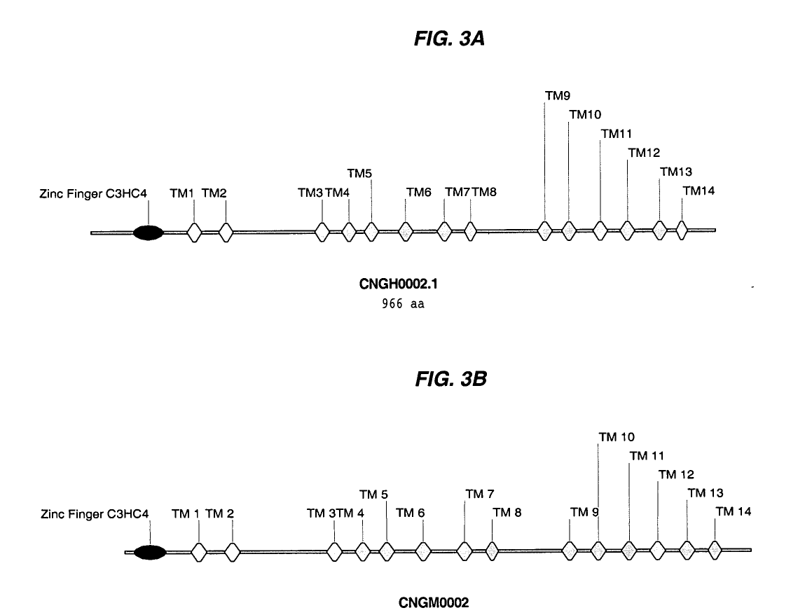

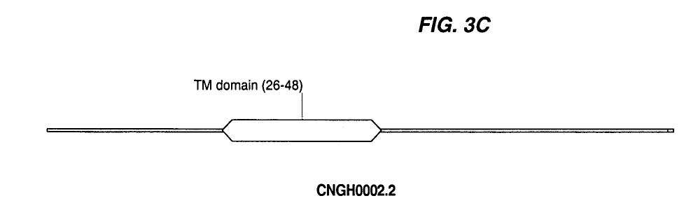

The protein encoded by CNGH0002.2 gene (a separate putative Open Reading Frame of the CNGH0002 gene) consists of 90 amino acids. It is predicted to be a transmembrane protein with a transmembrane region from residues 26-48 (FIG. 3C). A region of the protein, from amino acids 28-39, bears a similarity to M22 glycoprotease of the metallo-protease family.

### Gene Expression Pattern

The microarray data demonstrated that CNGH0002 gene expression was up-regulated by 1.7 fold during the peak of hypoxia treatment of MDA-MB 231 breast cancer cells, and was then down regulated during reoxygenation process, exhibiting the typical behavior of a hypoxia-response gene.

CNGH0002 transcript corresponds to 6 Unigene clusters: Hs.20141, Hs.102238, Hs.335134, Hs.314228, Hs.3808755, Hs.8078 (http://www.ncbi.nim.nih.gov/entrez/query.fcgi?db=unigene). These ESTs are distributed in a variety of tissues and organs, such as uterus, liver, spleen, retina, thyroid, heart, ovary, kidney, colon, placenta, skeletal muscle, and stomach. The pattern of expression is notable as appearing in many types of tumors, in particular insulinoma, ovarian tumor, glioblastoma, and adenocarcinoma.

In addition, several serial analysis of gene expression (SAGE) (http://www.ncbi.nlm.nih.gov/SAGE/) tags corresponding to sequences found in CNGH0002 showed elevated expression in ovarian carcinoma, metastatic mammary gland carcinoma, oligodendroglioma, pancreatic adenocarcinoma, and brain astrocytoma.

CNGH0002 proteins and nucleic acid molecules encoding them comprise a family of molecules having certain conserved structural and functional features. Each of these molecules is included in the invention. As used herein, the term “family” is intended to mean two or more proteins or nucleic acid molecules having a common or similar domain structure and having sufficient amino acid or nucleotide sequence identity as defined herein. Family members can be from either the same or different species. For example, a family can comprise two or more proteins of human origin, or can comprise one or more proteins of human origin and one or more of non-human origin.

A common domain present in CNGH0002 proteins is a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a CNGH0002 protein contains a signal sequence corresponding to amino acid residues 1 up to residue 35 of SEQ ID NO: 2. The signal sequence is cleaved during processing of the mature protein.

CNGH0002 proteins can include an extracellular domain. As used herein, an “extracellular domain” refers to a portion of a protein which is localized to the non-cytoplasmic side of a lipid bilayer of a cell when a nucleic acid encoding the protein is expressed in the cell. The human CNGH0002 protein extracellular domain is located from about amino acid residue 20 to about amino acid residue 147 of SEQ ID NO: 2.

In addition, CNGH0002 includes a transmembrane domain. As used herein, a “transmembrane domain” refers to an amino acid sequence which is at least about 20 to 25 amino acid residues in length and which contains at least about 65-70% hydrophobic amino acid residues such as alanine, leucine, phenylalanine, protein, tyrosine, tryptophan, or valine (Erik, et al. Proc. of Sixth Int. Conf. on Intelligent Systems for Molecular Biology, p 175-182). In a preferred embodiment, a transmembrane domain contains at least about 15 to 30 amino acid residues, preferably about 20-25 amino acid residues, and has at least about 60-80%, more preferably 65-75%, and more preferably at least about 70% hydrophobic residues. Thus, in one embodiment, an CNGH0002 protein of the invention contains one or more transmembrane domains corresponding to about amino acid residues 148 to 170 of SEQ ID NO: 2. CNGH0002 can alternately exist in a membrane-bound form having at least two transmembrane regions, wherein the protein has transmembrane domains from about amino acid residues 197 to 219, 346 to about 368, 388 to about 410, 423 to about 445, 476 to about 498, 536 to about 558, 578 to about 596, 691 to about 713, 728 to about 750, 777 to about 799, 819 to about 841, 869 to about 891, and from 906 to about 923 of SEQ ID NO: 2.

The present invention includes CNGH0002 proteins having a cytoplasmic domain, particularly including proteins having a carboxyl-terminal cytoplasmic domain. As used herein, a “cytoplasmic domain” refers to a portion of a protein which is localized to the cytoplasmic side of a lipid bilayer of a cell when a nucleic acid encoding the protein is expressed in the cell. The human CNGH0002 cytoplasmic domain is situated from about amino acid residue 924 to amino acid residue 966 of SEQ ID NO: 2 and Table 1.

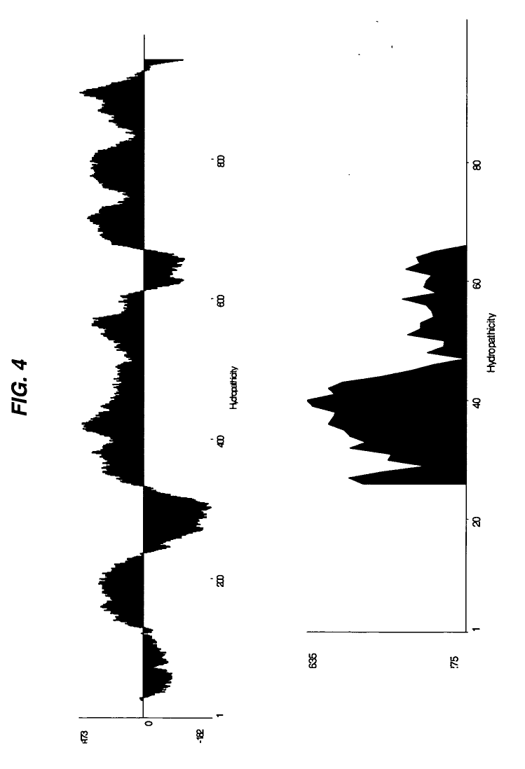

FIG. 4 depicts a hydrophobicity (or hydrophilicity) plot of human CNGH0002 protein. Relatively hydrophobic regions are above the dashed horizontal line, and relatively hydrophilic regions are below the dashed horizontal line using the Kyte-Doolittle method (J. Mol. Biol. (1982) 157, 105-132). Relatively hydrophilic regions are generally located at or near the surface of a protein, and are more frequently effective immunogenic epitopes than are relatively hydrophobic regions. For this reason, such plots are termed hydropathicity plots. For example, regions of human CNGH0002 protein located at or near the surface of the protein and those predicted to be embedded in the membrane are shown in Table 3.

TABLE 3

| | AA Residue | AA Residue |
|---|---|---|
| Region | Start | Stop |
| outside | 1 | 147 |
| TM 1 | 148 | 170 |
| inside | 171 | 198 |
| TM 2 | 197 | 219 |
| inside | 210 | 345 |
| TM 3 | 346 | 368 |
| outside | 369 | 387 |
| TM 4 | 388 | 410 |
| inside | 411 | 422 |
| TM 5 | 423 | 445 |
| outside | 446 | 475 |
| TM 6 | 476 | 498 |
| inside | 499 | 535 |
| TM 7 | 536 | 558 |
| outside | 559 | 577 |
| TM 8 | 578 | 596 |
| inside | 597 | 690 |
| TM 9 | 691 | 713 |
| outside | 714 | 727 |
| TM 10 | 728 | 750 |
| inside | 751 | 776 |
| TM 11 | 777 | 799 |
| outside | 800 | 818 |
| TM 12 | 819 | 841 |
| inside | 842 | 868 |
| TM 13 | 869 | 891 |
| outside | 892 | 905 |
| TM 14 | 906 | 923 |
| inside | 924 | 966 |

CNGH0002 proteins typically comprise a variety of potential post-translational modification sites (often within an extracellular domain), such as those predicted by computerized sequence analysis of CNGH0002 proteins using amino acid sequence comparison software (comparing the amino acid sequence of CNGH0002 with the information in the PROSITE database (rel. 12.2; February, 1995) and the Hidden Markov Models database (Rel. PFAM 3.3)). In certain embodiments, a protein of the invention has at least one of the post-translational modification sites shown in FIG. 4B.

CNGH0002 exhibits no significant amino acid sequence similarity or nucleic acid sequence similarity with any known protein or nucleic acid sequence. Thus, CNGH0002 appears to be a novel protein and nucleic acid.

### Biological Function of CNGH0002 Proteins, Nucleic Acids Encoding them, and Modulators of these Molecules

CNGH0002 proteins are involved in disorders which affect both tissues in which they are normally expressed and tissues in which they are normally not expressed. CNGH0002 is expressed in endothelial cells, and is thus involved in one or more physiological processes which occur in blood vessels. Such processes include, for example, stress response such as those to injury or oxidative damage after ischemia and reperfusion which may include the release of cytokines, chemokines, growth factors, and proteases. The proteins encoded by the hypoxia-responsive CNGH0002 proteins play a role in cancer, ischemia, reperfusion, retinopathy, neonatal distress, preeclampsia, cardiac arrest, stroke, wound healing, other cardiovascular diseases, Alzheimer's disease and pulmonary diseases.

Clones encoding human CNGH0002.1 and CNGH0002.2 proteins were deposited with ATCC. This deposit will be maintained under the terms of the Budapest Treaty on the International Recognition of the Deposit of Microorganisms for the Purposes of Patent Procedure.

Various aspects of the invention are described in further detail in the following subsections.

I. Isolated Nucleic Acid Molecules

The invention includes nucleic acid molecules which encode a polypeptide of the invention. Such nucleic acids include, for example, a DNA molecule having the nucleotide sequence listed in SEQ ID NO: 1 or some portion thereof, such as the portion which encodes mature CNGH0002 protein, immature CNGH0002 protein, or a domain of CNGH0002 protein. These nucleic acids are collectively referred to as nucleic acids of the invention.

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of all or a portion of SEQ ID NOs: 1, 6, 12, or 14, or a complement thereof, or which has a nucleotide sequence comprising one of these sequences, can be isolated using standard molecular biology techniques and the sequence information provided herein. Using all or a portion of the nucleic acid sequences of SEQ ID NOs: 1, 6, 12, or 14 as a hybridization probe, nucleic acid molecules of the invention can be isolated using standard hybridization and cloning techniques (e.g., as described in Sambrook et al., eds., Molecular Cloning: A Laboratory Manual, 2 nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of SEQ ID NOs: 1, 2, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 15, preferably about 25, more preferably about 50, 75, 100, 125, 150, 175, 200, 250, 300, 350, or 400 or more consecutive nucleotides of the sense or anti-sense sequence of one of SEQ ID NOs: 1, 2, or of a naturally occurring mutant of one of SEQ ID NOs: 1 and 2.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of SEQ ID NO: 2, expressing the encoded portion of the polypeptide protein (e.g., by recombinant expression in vitro), and assessing the activity of the encoded portion of the polypeptide.

The invention further encompasses nucleic acid molecules that differ from the nucleotide sequence of SEQ ID NOs: 1 or 2 due to degeneracy of the genetic code and thus encode the same protein as that encoded by the nucleotide sequence of SEQ ID NO: 2.

In addition to the nucleotide sequences of SEQ ID NO: 2, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus.

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the protein described herein are intended to be within the scope of the invention. Nucleic acid molecules corresponding to natural allelic variants and homologs of a cDNA of the invention can be isolated based on their identity to human nucleic acid molecules using the cDNAs, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 15 (25, 40, 60, 80, 100, 150, 200, 250, 300, 350, 400, 450, 550, 650, 700, 800, 900, 1000, 1200, or 1232) nucleotides in length and hybridizes under stringent conditions to the nucleic acid molecule comprising the nucleotide sequence, preferably the coding sequence, of SEQ ID NOs: 1 or 2, or a complement thereof. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NOs: 1 or 2, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that can exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from SEQ ID NOs: 2-5, 7, 8, 13, OR 15, yet retain biological activity. In one embodiment, the isolated nucleic acid molecule includes a nucleotide sequence encoding a protein that includes an amino acid sequence that is at least about 40% identical, 50%, 60%, 70%, 80%, 90%, 95%, or 98% identical to the amino acid sequence of one of SEQ ID NOs: 2-5, 7, 8, 13, OR 15.

An isolated nucleic acid molecule encoding a variant protein can be created by introducing one or more nucleotide substitutions, additions or deletions into the nucleotide sequence of SEQ ID NOs: 1, 6, 12, OR 14, such that one or more amino acid residue substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e. g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein:protein interactions with the polypeptide of the invention; (2) the ability to bind a ligand of the polypeptide of the invention (e.g. another protein identified herein); (3) the ability to bind to a modulator or substrate of the polypeptide of the invention; or (4) the ability to modulate a physiological activity of the protein, such as one of those disclosed herein.

The present invention encompasses antisense nucleic acid molecules, i. e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses ribonucleic acid molecules which are complementary, antisense, double stranded homologues, siRNA, or are sequence specific single-stranded RNAs which form short hairpin structures, shRNA, that can be used to down-modulate specific gene expression, in this case, CNGH0002.1 or CNGH0002.2, and therefore to inhibit protein expression and to elucidate their respective biological functions. (Fire, A., et al. (1998) Nature 391: 806-811; Paddison, P. J. et al. (2002) Genes Develop 16:948-958).

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e. g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a step-wise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

II. Isolated Proteins and Antibodies

A. Proteins

The invention thus includes purified human CNGH0002 protein, both in the form of the immature amino acid residue protein and in the form of the mature protein. Mature human CNGH0002 protein can be synthesized without the signal sequence polypeptide at the amino terminus thereof, or it can be synthesized by generating immature CNGH0002 protein and cleaving the signal sequence therefrom.

It is furthermore recognized that CNGH0002 can exist, in a membrane bound form, wherein the protein has at least one transmembrane region as shown in Table 1.

In addition to full length mature and immature human CNGH0002 proteins, the invention includes fragments, derivatives, and variants of these CNGH0002 proteins, as described herein. These proteins, fragments, derivatives, and variants are collectively referred to herein as polypeptides of the invention or proteins of the invention.

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in any of SEQ ID NOs: 2-5, 7, 8, 13, or 14), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of one of SEQ ID NOs: 2-5, 7, 8, 13, or 14. Other useful proteins are substantially identical (e.g., at least about 40%, preferably 50%, 60%, 70%, 80%, 90%, 95%, or 99%) to any of SEQ ID NOs: 2-5, 7, 8, 13, or 14 and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov (e.g., BLOSUM62 residue wight matrix, gap existence cost 11, per residue gap cost 1, and lambda ratio 0.85). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention. In another embodiment, a polypeptide or the invention or a domain or active fragment thereof can be fused with a heterologous protein sequence or fragment thereof to form a chimeric protein, where the invention polypeptides, domains or fragments are not fused end to end but are interposed within the heterologous protein framework.

One useful fusion protein is a GST fusion protein in which the polypeptide of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. A preferred embodiment of a immunoglobulin chimeric protein is a CH1 domain-deleted immunoglobulin or “mimetibody” having an active polypeptide fragment interposed within a modified framework region as taught in applicants co-pending application U.S. Ser. No. 60/392,431. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e. g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention (e.g. the signal sequence in SEQ ID NOs: 2-5, 7, 8 13, or 15) can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

In another embodiment, the signal sequences of the present invention can be used to identify regulatory sequences, e.g., promoters, enhancers, repressors. Since signal sequences are the most amino-terminal sequences of a peptide, the nucleic acids which flank the signal sequence on its amino-terminal side are likely regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

### B. Antibodies

The present invention further includes, but is not limited to, methods of using the nucleic acids and polypeptides encoded thereby to make antibodies and anti-idiotype antibodies, including diagnostic and therapeutic compositions, methods and devices. Such antibodies optionally further affect a specific ligand, such as but not limited to where such antibody modulates, decreases, increases, antagonizes, agonizes, mitigates, aleviates, blocks, inhibits, abrogates and/or interferes with at least one CNGH0002 activity or binding, or with CNGH0002 receptor activity or binding, in vitro, in situ and/or in vivo. As a non-limiting example, a suitable CNGH0002 antibody, specified portion or variant of the present invention can bind at least one CNGH0002 protein, or specified portions, variants or domains thereof. A suitable CNGH0002 antibody, specified portion, or variant can also optionally affect at least one of CNGH0002 activity or function, such as but not limited to, RNA, DNA or polypeptide synthesis, CNGH0002 release, CNGH0002 receptor signaling, membrane CNGH0002 cleavage, CNGH0002 activity, CNGH0002 production and/or synthesis. CNGH0002 antibodies useful in the methods and compositions of the present invention can optionally be characterized by high affinity binding to CNGH0002 and optionally and preferably having low toxicity

As used herein, a “CNGH0002 antibody,” and the like include any polypeptide or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion, fragment or variant thereof, or at least one portion of an CNGH0002 receptor or binding polypeptide, which can be incorporated into a CNGH0002 antibody of the present invention.

Antibodies can include one or more of at least one CDR, at least one variable region, at least one constant region, at least one heavy chain (e.g., g1, g2, g3, g4, m, a1, a2, d, e), at least one light chain (e.g., k and I), or any portion or fragment thereof, and can further comprise interchain and intrachain disulfide bonds, hinge regions, glycosylation sites that can be separated by a hinge region, as well as heavy chains and light chains. Light chains typically have a molecular weight of about 25 Kd and heavy chains typically range from 50K-77 Kd. Light chains can exist in two distinct forms or isotypes, kappa (k) and lambda (I), which can combine with any of the heavy chain types. All light chains have at least one variable region and at least one constant region. The IgG antibody is considered a typical antibody structure and has two intrachain disulfide bonds in the light chain (one in variable region and one in the constant region), with four in the heavy chain, and such bond encompassing a peptide loop of about 60-70 amino acids comprising a “domain” of about 110 amino acids in the chain. IgG antibodies can be characterized into four classes, IgG1, IgG2, IgG3 and IgG4. Each immunoglobulin class has a different set of functions. The following table summarizes the Physicochemical properties of each of the immunoglobuling classes and subclasses.

| Property | IgG1 | IgG2 | IgG3 | IgG4 | IgM | IgA1 | IgA2 | SIgA | IgD | IgE |
|---|---|---|---|---|---|---|---|---|---|---|
| Heavy | γ1 | γ1 | γ1 | γ1 | μ | α1 | α2 | α1/α2 | δ | e |
| Chain | | | | | | | | | | |
| Mean | 9 | 3 | 1 | 0.5 | 1.5 | 3.0 | 0.5 | 0.05 | 0.03 | 0.00005 |
| Serum | | | | | | | | | | |
| conc. | | | | | | | | | | |
| (mg/ml) | | | | | | | | | | |
| Sedimentation | 7s | 7s | 7s | 7s | 19s | 7s | 7s | 11s | 7s | 8s |
| constant | | | | | | | | | | |
| Mol. Wt. | 146 | 146 | 170 | 146 | 970 | 160 | 160 | 385 | 184 | 188 |
| (×10³) | | | | | | | | | | |
| Half Life | 21 | 20 | 7 | 21 | 10 | 6 | 6 | ? | 3 | 2 |
| (days) | | | | | | | | | | |
| % | 45 | 45 | 45 | 45 | 80 | 42 | 42 | Trace | 75 | 50 |
| intravascular | | | | | | | | | | |
| distribution | | | | | | | | | | |
| Carbohydrate (%) | 2-3 | 2-3 | 2-3 | 2-3 | 12 | 7-11 | 7-11 | 7-11 | 9-14 | 12 |

The following table summariizes non-limiting examples of antibody effector functions for human antibody classes and subclasses.

| Effector | | | | | | | | |
|---|---|---|---|---|---|---|---|---|
| function | IgG1 | IgG2 | IgG3 | lgG4 | IgM | IgA | IgD | IgE |
| Complement | . | . | . | | . | | | − |
| fixation | | | | | | | | |
| Placental | + | + | + | + | − | − | − | − |
| transfer | | | | | | | | |
| Binding to | +++ | +++ | − | +++ | − | − | − | − |
| Staph A | | | | | | | | |
| Binding to | +++ | +++ | +++ | +++ | − | − | − | − |
| Strep G | | | | | | | | |

Accordingly, the type of antibody or fragment thereof can be selected for use according to the present invention based on the desired characteristics and functions that are desired for a particular therapeutic or diagnostic use, such as but not limited to serum half life, intravascular distribution, complement fixation, etc.

The isolated nucleic acids of the present invention can be used for production of at least one CNGH0002 antibody or specified variant thereof, which can be used to measure or effect in an cell, tissue, organ or animal (including mammals and humans), to diagnose, monitor, modulate, treat, alleviate, help prevent the incidence of, or reduce the symptoms of, at least one CNGH0002 condition, selected from, but not limited to, at least one of an immune disorder or disease, a cardiovascular disorder or disease, an infectious, malignant, and/or neurologic disorder or disease, or other known or specified CNGH0002 related condition.

Such a method can comprise administering an effective amount of a composition or a pharmaceutical composition comprising at least one CNGH0002 antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 mg/ml serum concentration per single, multiple, or continuous adminstration, or any effective range or value therein, as done and determined using known methods, as described herein or known in the relevant arts.

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of SEQ ID NOs: 2, 3, 5, 7, or 9 and encompasses an epitope of the protein such that an antibody raised against the peptide forms a specific immune complex with the protein.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Antibody producing cells can be obtained from the peripheral blood or, preferably the spleen or lymph nodes, of humans or other suitable animals that have been immunized with the immunogen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

In one approach, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, or heteromylomas, fusion products thereof, or any cell or fusion cell derived therefrom, or any other suitable cell line as known in the art. See, e.g., www.atcc.org, www.lifetech.com., and the like, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Other suitable methods of producing or isolating antibodies of the requisite specificity can be used, including, but not limited to, methods that select recombinant antibody from a peptide or polypeptide library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; Biolnvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or polypeptides—U.S. Pat. Nos. 5,723,323, 5,763,192, 5,814,476, 5,817,483, 5,824,514, 5,976,862, WO 86/05803, EP 590 689 (Ixsys, now Applied Molecular Evolution (AME), each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source which is non-human, e.g., but not limited to mouse, rat, rabbit, non-human primate or other mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html; www.sciquest.com/; www.abcam.com/; www.antibodyresource.com/onlinecomp.html; www.public.iastate.edu/˜pedro/research_tools.html; www.mgen.uni-heidelberg.de/SD/IT/IT.html; www.whfreeman.com/immunology/CH05/kuby05.htm; www.library.thinkquest.org/12429/Immune/Antibody.html; www.hhmi.org/grants/lectures/1996/vlab/; www.path.cam.ac.uk/˜mrc7/mikeimages.html; www.antibodyresource.com/;

mcb.harvard.edu/BioLinks/Immunology.html.www.immunologylink.com/; pathbox.wustl.edu/˜hcenter/index.html; www.biotech.ufl.edu/˜hcl/;

www.pebio.com/pa/340913/340913.html; www.nal.usda.gov/awic/pubs/antibody/;

www.m.ehime-u.ac.jp/˜yasuhito/Elisa.html; www.biodesign.com/table.asp;

www.icnet.uk/axp/facs/davies/links.html; www.biotech.ufl.edu/˜fccl/protocol.html; www.isac-net.org/sites_geo.html; aximt1.imt.uni-marburg.de/˜rek/AEPStart.html; baserv.uci.kun.nl/˜jraats/links1.html; www.recab.uni-hd.de/immuno.bme.nwu.edu/; www.mrc-cpe.cam.ac.uk/imt-doc/public/INTRO.html; www.ibt.unam.mx/vir/V_mice.html; imgt.cnusc.fr:8104/; www.biochem.ucl.ac.uk/˜martin/abs/index.html; antibody.bath.ac.uk/;

abgen.cvm.tamu.edu/lab/wwwabgen.html; www.unizh.ch/˜honegger/AHOseminar/Slide01.html; www.cryst.bbk.ac.uk/˜ubcg07s/; www.nimr.mrc.ac.uk/CC/ccaewg/ccaewg.htm; www.path.cam.ac.uk/˜mrc7/humanisation/TAHHP.html; www.ibt.unam.mx/vir/structure/stat_aim.html; www.biosci.missouri.edu/smithgp/index.html; www.cryst.bioc.cam.ac.uk/˜fmolina/Web-pages/Pept/spoffech.html; www.jerini.de/fr_products.htm; www.patents.ibm.com/ibm.html.Kabat et al., Sequences of Polypeptides of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. Generally part or all of the non-human or human CDR sequences are maintained while the non-human sequences of the variable and constant regions are replaced with human or other amino acids. antibodies can also optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; each entirely incorporated herein by reference, included references cited therein.

The CNGH0002 antibody can also be optionally generated by immunization of a transgenic animal (e.g., mouse, rat, hamster, non-human primate, and the like) capable of producing a repertoire of human antibodies, as described herein and/or as known in the art. Cells that produce a human CNGH0002 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 Al, Surani et al. US. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Once obtained, production of the antibodies of the present invention can also be prepared using at least one CNGH0002 antibody encoding nucleic acid to provide transgenic animals or mammals, such as goats, cows, horses, sheep, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference. Antibodies of the present invention can additionally be prepared using at least one CNGH0002 antibody encoding nucleic acid to provide transgenic plants and cultured plant cells (e.g., but not limited to tobacco and maize) that produce such antibodies, specified portions or variants in the plant parts or in cells cultured therefrom.

The antibodies of the invention can bind human CNGH0002with a wide range of affinities (K_{D}). In a preferred embodiment, at least one human mAb of the present invention can optionally bind human CNGH0002 with high affinity. For example, a human mAb can bind human CNGH0002with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion and chimeric proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. To assist in affinity purification, various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol, 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Enoineering 3(6):547-553 (1990)). Other tag polypeptides include the Flag-peptide (Hopp et al., Bio Technology, 6:1204-1210 (1988)); the KT3 epitope peptide (Martin et al., Science, 255:192-194 (1992)); an .alpha.-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)). A preferred tag is the FLAG tag.

Examples of suitable inducible non-fusion E. coli expression vectors include pTrc (Amann et al., (1988) Gene 69:301-315) and pET 11 d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident A prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSecl (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells). A number of suitable mammalian host cell lines capable of expressing intact glycosylated polypeptides have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org).

Expression vectors for these cells can include one or more of the following expression control sequences, a promoter, an enhancer, and/or processing information sites, such as ribosome binding sites, RNA splice sites, polyadenylation sites, and transcriptional terminator sequences (See, e.g., Ausubel et al., supra; Sambrook, et al., supra).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In another aspect, the invention relates to CNGH0002 polypeptides or antibodies of the invention, as described herein, which are modified by the covalent attachment of a moiety. Such modification can produce a CNGH0002 polypeptide or anibody with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms. As used herein, the term “fatty acid” encompasses mono-carboxylic acids and di-carboxylic acids. Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-delta 9-octadecanoate (C₁₈, oleate), all cis-delta5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human polypeptides and antibodies can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation or buccal), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin. Pharmaceutical excipients and additives useful in stabilizing the present composition include but are not limited to polypeptides, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary but non-limiting polypeptide excipients include serum albumin such as human serum albumin (HSA), recombinant human albumin (rHA), gelatin, casein, and the like. Representative amino acid, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosolized particles from a pressurized container or dispenser which contains a suitable propellant, e.g., a gas such as carbon dioxide, or a nebulizer. Alternatively, compositions formulated as particles can be dispersed by electrostatic, mechanical means including vibrations, or ultrasonic means as taught in U.S. Pat. No: U.S. Pat. No. 4,530,464; U.S. Pat. No. 4,533,082; U.S. Pat. No. 5,838,350; U.S. Pat. No. 6,113,001; U.S. Pat. No. 6,514,496; U.S. Pat. No. 5,518,179; U.S. Pat. No. 5,152,456; U.S. Pat. No. 5,261,601; AND U.S. Pat. No. 4,605,167.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes having monoclonal antibodies incorporated therein or thereon) can also be used as pharmaceutically acceptable carriers. Particularly preferred compositions and methods are taught in U.S. Pat. Nos. 5,891,468 and 6,316,024.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologs, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used for all of the purposes identified herein in portions of the disclosure relating to individual types of protein of the invention (e.g. CNGH0002 protein). The isolated nucleic acid molecules of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention can be used to screen drugs or compounds which modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect and isolate a protein of the invention or to modulate activity of a protein of the invention.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), glass slides also known as “gene chips” (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, the assay involves assessment of an activity characteristic of the polypeptide, wherein binding of the test compound with the polypeptide or a biologically active portion thereof alters (i.e. increases or decreases) the activity of the polypeptide.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide to bind to or interact with a target molecule or to transport molecules across the cytoplasmic membrane.

Determining the ability of a polypeptide of the invention to bind to or interact with a target molecule can be accomplished by one of the methods described above for determining direct binding. As used herein, a “target molecule” is a molecule with which a selected polypeptide (e.g., a polypeptide of the invention binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., an mRNA, intracellular Ca²⁺, diacylglycerol, IP3, and the like), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic activity, the enzymatic activity, or both, of the target molecule on an appropriate substrate can be determined as previously described.

In yet another embodiment, the cell-free assay comprises contacting a polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it can be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl-N,N-dimethyl-3-ammonio-1-propane sulfonate.

In one or more embodiments of the above assay methods of the present invention, it can be desirable to immobilize either the polypeptide of the invention or its target molecule to facilitate separation of complexed from non-complexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione Sepharose beads (Sigma Chemical; St. Louis, Mo.) or glutathione derivatized microtiter plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (i.e. statistically significantly greater) in the presence of the candidate compound than in its absence, the candidate compound is identified as a stimulator of the selected mRNA or protein expression. Alternatively, when expression of the selected mRNA or protein is less (i.e. statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sub-localization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al. (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences of SEQ ID NO: 1 can comfortably provide positive individual identification with a panel of perhaps 10 to 1,000 primers which each yield a non-coding amplified sequence of 100 bases. If a predicted coding sequence, such as that in SEQ ID NO: 1 from position 16 to 2916 or 6344 to 6616 is used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

3. Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to non-coding regions are particularly appropriate for this use as greater numbers of polymorphisms occur in the non-coding regions, making it easier to differentiate individuals using this technique. Examples of polynucleotide reagents include the nucleic acid sequences of the invention or portions thereof, e.g., fragments derived from non-coding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

Another aspect of the invention provides methods for expression of a nucleic acid or polypeptide of the invention or activity of a polypeptide of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of a polypeptide of the invention in clinical trials. These and other agents are described in further detail in the following sections.

1. Diagnostic Assays

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NO: 1 or a portion thereof, such as an oligonucleotide of at least 15, 30, 50, 100, 250 or 500 nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a polypeptide of the invention is an antibody capable of binding to a polypeptide of the invention, preferably an antibody with a detectable label. Antibodies can be polyclonal, or more preferably, monoclonal. An intact antibody, or a fragment thereof (e. g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a polypeptide of the invention or mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (e. g., one of the disorders described in the section of this disclosure wherein the individual polypeptide of the invention is discussed). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a polypeptide of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

2. Prognostic Assays

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention (e.g., one of the disorders described in the section of this disclosure wherein the individual polypeptide of the invention is discussed). Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. PCR and/or LCR can be desirable to use as a preliminary amplification step in conjunction with any of the techniques used for detecting mutations described herein.

Alternative amplification methods include: self-sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. RNA/DNA duplexes can be treated with RNASE to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called DNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) can be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp’ of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization; Gibbs et al. (1989) Nucleic Acids Res. 17:2437-2448) or at the extreme 3′ end of one primer where, under appropriate conditions, mismatching can prevent or reduce polymerase extension (Prossner (1993) Tibtech 11:238). In addition, it can be desirable to introduce a novel restriction site in the region of the mutation to create cleavage-based detection (Gasparini et al. (1992) Mol. Cell Probes 6:1). Amplification can also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a gene encoding a polypeptide of the invention. Furthermore, any cell type or tissue, preferably peripheral blood leukocytes, in which the polypeptide of the invention is expressed can be utilized in the prognostic assays described herein.

3. Pharmacogenomics

Agents, or modulators which have a stimulatory or inhibitory effect on activity or expression of a polypeptide of the invention as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

4. Monitoring of Effects During Clinical Trials

Monitoring the influence of agents (e.g., drug compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels, or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state can be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent can be desirable to increase the expression or activity of the polypeptide to higher levels than detected, i.e., to increase the effectiveness of the agent. Alternatively, decreased administration of the agent can be desirable to decrease expression or activity of the polypeptide to lower levels than detected, i.e., to decrease the effectiveness of the agent.

C. Methods of Treatment

The present invention provides for both prophylactic and therapeutic methods of treating a subject at risk of (or susceptible to) a disorder or having a disorder associated with aberrant expression or activity of a polypeptide of the invention and/or in which the polypeptide of the invention is involved. Disorders characterized by aberrant expression or activity of the polypeptides of the invention are described elsewhere in this disclosure.

1. Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

2. Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a polypeptide of the invention. In one embodiment, the method involves administering an agent (e.g., an agent identified by a screening assay described herein), or combination of agents that modulates (e.g., up-regulates or down-regulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low or down-regulated and/or in which increased activity is likely to have a beneficial effect, e.g., in wound healing. Conversely, inhibition of activity is desirable in situations in which activity or expression is abnormally high or up-regulated and/or in which decreased activity is likely to have a beneficial effect.

### EXAMPLES

The following specific examples are intended to illustrate the invention and should not be construed as limiting the scope of the claim.

### Example 1

### Identification of CNGH0002 by Microarray Analysis

### I. In Vitro Tumor Hypoxia/Re-Oxygenation Model

MDA-MB 231, human breast carcinoma cells, were obtained from ATCC (Manassas, Va.) and were maintained at Cell Biology Service of Centocor. Cells were cultured under standard conditions recommended by the supplier. Briefly, proliferating cells were cultured in Dulbecco's Modified Eagle Medium (DMEM, Gibco BRL Grand Island, N.Y.) containing 10% calf serum and 1% L-Glutamine, Penicillin G, Streptomycin Sulfate (GPS, Gibco BRL Grand Island, N.Y.). For hypoxic culturing, 80% confluent cells were placed in airtight chambers (BellCo, Vineland, N.J.) equilibrated with pre-calibrated air of 0.5% O2, 5% CO2, and 94% N2 (Northeast Airgas, Radnor, Pa.) in a humidified atmosphere at 370° C. for 4, 8, 12, 16, 20 and 24 hours. For re-oxygenation experiments, cells exposed to hypoxic stress for 24 hours were transferred to normoxic culturing environments for 4, 8, 12, 16, 20 and 24 hours.

### II. RNA Sample Preparation and Microarray Hybridization

Cells were cultured under normoxic, hypoxic, and re-oxygenation conditions for different time periods. Total RNA was extracted from these cells using TRIZol RNA Extracting Reagent according to manufacturer's instruction (Gibco BRL, Grand Island, N.Y.). Total RNA samples were hybridized to two different types of cDNA arrays produced in Johnson & Johnson Pharmaceutical R & D at La Jolla. RNA preparation, labeling, and hybridization were performed as reported previously (13).

### III. Data Analysis and Identification of CNGH0002, a Novel Hypoxia-Response Gene

Raw intensity data from the cDNA arrays were first normalized within each sample. Linear normalization and then nonlinear normalization was performed within each sample. Outlier intensity data points (greater than 1.4 fold away from the median of replicate measurements) were identified and removed from the data sets. Average intensity was determined by calculating the arithmatic mean of nonoutlier intensity values. Spline normalization of the average intensity was then performed across all samples in the data sets. Raw intensity data was imported from internal data server into OmniViz software (Maynard, Mass.). Gene expression comparison at different time points during normoxia, hypoxia, and reoxygenation was expressed as ratios of expression levels. Log2 of ratios were further used for cluster analysis using the K-means method. All genes were filtered by a single fold change in expression levels greater than or equal to 2 as compared to that under normoxia conditions. Qualified genes were subsequently clustered using a hierarchical method and correlation metric.

Gene expression profiling analysis not only provided information regarding gene expression in these cancer cells under normoxia verses hypoxia conditions, but will also elucidate the dynamics of changes in gene expression during the transitions from normoxia, hypoxia, reoxygenation conditions. Hypoxia response genes are defined as those genes whose expression level increases when cells were subjected to hypoxia stress, and the expression level decreases in cells under re-oxygenation conditions.

### Example 2

### Determination of CNGH0002 Gene Expression in Human Cancer Cell Lines by Northern Blot Analysis

Northern blots containing 2 μg of poly A⁺ RNA extracted from eight different human cancer cell lines were obtained from Clontech (Palo Alto, Calif.). The blots were hybridized in Express Hybridization Solution (Clontech, Palo Alto, Calif.) at 42° C. containing ³²P-labeled CNGH0002-specific cDNA probes. After overnight hybridization, the blots were washed twice in 2×SSC/0.1% SDS, twice in 0.1×SSC/0.1% SDS at 42° C., and were exposed overnight in a Phosphor Screen exposure cassette and scanned using STORM Scanner, Typhooon 8600 (Molecular Dynamics, Amersham Pharmacia Biotech, Piscataway, N.J.). The band intensity of the scanned images was quantified using ImageQuant 5.2 software (Molecular Dynamics, Amersham Pharmacia Biotech, Piscataway, N.J.) and relative expression level of CNGH0002 were presented as volume report (Table 4). Northern blotting analysis demonstrated that CNGH0002 is expressed in a variety of human tumor cell lines, with high expression levels in HeLa S3 ovarian cancer cells, K-562 chronic myelogenous leukemia cells, SW480 colorectal cancer cells, and G361 melanoma cells.

TABLE 4
Gene expression of CNGH0002 in different human cancer cell lines

| | Expression Level |
|---|---|
| Cancer Cell Lines | (Arbitrary Units) |
| Promyelocytic leukemia HL-60 | 15717 |
| Ovarian cancer HeLa S3 | 99848 |
| Chronic myelogenous leukemia K-562 | 34373 |
| Lymphoblastic leukemia MOLT-4 | 10209 |
| Burkitt's lymphoma Raji | 14419 |
| Colorectal adenocarcinoma SW480 | 34129 |
| Lung carcinoma A549 | 13435 |
| Melanoma G-361 | 28777 |

### Example 3

### Isolation and Analysis of Full Length cDNA Sequence

The full-length cDNA sequences of two putative open-reading frames (ORF) encoded by CNGH0002.1 and CNGH0002.2 genes were amplified using reverse transcription-polymerase chain reaction (RT-PCR). The sequence of PCR primers used for RT-PCR-based amplification and cloning are the following: Six PCR primers were designed for cloning the full-length ORF of CNGH0002.1. They are Boston Large FOR 5′-CATATGTAGA GTGTGTCGGTCAGAAGGAAC-3′bp 204-233 direct (SEQ ID NO: 10), Boston Large REV 5′-GTGGAGATGAGCCTTGTTTG-3′ bp 2871-2895 (SEQ ID NO: 11); Boston Small FOR 5′-GATGCTAATA ACGGAGCCCA GGATG-3′ bp 928-952 direct (SEQ ID NO: 12), Boston Small REV 5′-TTCTCCCACC ACAGGAATAGCGTTG-3′ bp 1965-1989 antisense (SEQ ID NO: 13); Boston 1st Exon FOR 5′-GATGCTAATA ACGGAGCCCA GGATG-3′ (SEQ ID NO: 14), and Boston 1st Exon REV 5′-TGCAGGACTCCAAGTGCCCA-3′ bp 2470-2489 antisense (SEQ ID NO: 15). The CNGH0002.2 ORF was cloned using Forward primer 5 5′-ACACGTGCAG GAGMTGTAG3′bp 6362-6382 direct (SEQ ID NO: 16) and Reverse primer 5 5′-GGTAAGACAT TTTAGTTGGG-3′bp 6555-6574 antisense (SEQ ID NO: 17).

### Cloning of CNGH0002.1 ORF

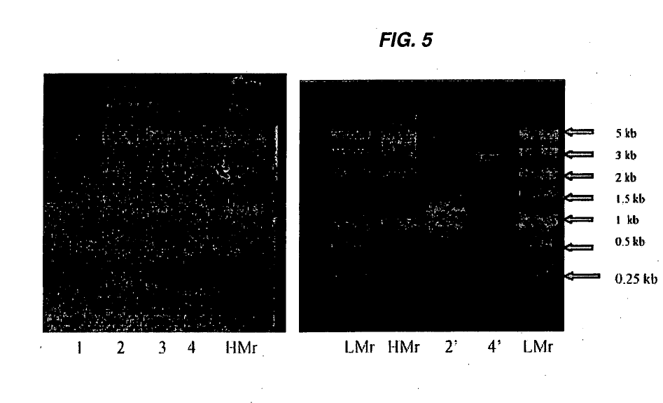

First, cDNA of colon cancer and breast (mammary) cancer was screened for the presence of CNGH0002.1 gene using Boston Small FOR and Boston Small REV primers. PCR reactions were performed in 50 ml volume containing 1× TAQMAN buffer A, 5.5 mM of magnesium chloride, 200 mM of dATP, dCTP, dGTP, and 400 mM of dUTP, 450 nM of the probe, 900 nM of each of the primers, 0.01 U/ml of AmpErase UNG, 0.025 U/ml of AmpliTaqGold DNA Polymerase, and 2 ng of cDNA in Rnase-free water. All PCR reagents were obtained from Applied Biosystems, Foster City, Calif.). The PCR reaction was then performed at 50° C. for 2 minutes followed by 95° C. for 10 min and then 40 cycles of 95° C. for 15 seconds and 60° C. for 1 minute. As shown in FIG. 5, amplicons with molecular size of approximately 1.06 kB were amplified from both colon cancer cDNA and breast cancer cDNA, confirming the existence of CNGH0002.1 gene in these cancer samples. We then attempted to amplify a large portion of the CNGH0002.1 ORF using primers Boston Large FOR and Boston Large REV. This time, we were only able to amplify a very small amount of amplicon from breast cancer cDNA, but not from colon cancer cDNA. The amplicon has an expected molecular size of approximately 2.7 kb.

To clone these amplicons, we carried out a second round PCR amplification reactions in which the amplicons from the first round PCR reactions were used as templates. Reamplification of the small and large amplicons from breast cancer resulted in a significant increase in the amount of PCR products and greatly facilitated the cloning process. As shown in FIG. 5, two clear bands of ˜1.06 kb and ˜2.7 kb were successfully amplified. These two PCR products were subsequently cloned into pcDNA3.1 vector (Invitrogen). The large PCR product represents exons 2 to 26 of CNGH0002.1 gene.

To clone the remaining 1st exon of CNGH0002.1 gene, Boston 1st Exon FOR 5′- and Boston 1st Exon REV primers were used.

These PCR products were sequence verified and were confirmed to represent the cDNA sequence of predicted CNGH0002.1 ORF as in SEQ ID NO: 1 from position 16 to 2916 (FIG. 5).

### Example 4

### Confirmation of Hypoxia Regulated CNGH0002 Gene Expression by Quantitative Real-Time PCR

Gene expression level of CNGH0002 under hypoxia, normoxia and reoxygenation was quantitatively determined by real-time reverse transcription polymerase chain reaction (RT-PCR/TAQMAN) analysis. CNGH0002 primers (CNGH0002For and CNGH0002Rev) and probe (CNGH0002-p) were designed using Primer Express software and synthesized by Invitrogen (Carlsbad, Calif.) and Synthegen (Houston, Tex.) respectively. Primer CNGH0002For (sense) corresponds to nucleic acids 6403-6427 of SEQ ID NO: 1 with a sequence of 5′-TTTTAAAACTTTCCAAGCTAGCTAC-3′ (SEQ ID NO: 18). Primer CNGH0002Rev (anti-sense) spans nucleic acids 6473-6502 of SEQ ID NO1 with a sequence of 5′-TTTAATTTTACAATGTAGAAAACTTCTCAC-3′ (SEQ ID NO:19). Probe CNGH0002-p1 is composed of nucleic acids 6434-6463 of SEQ ID NO1 with a sequence of 5′-FAM-TCATTTTCAGGGTTGAGTACTCTAAGCTCT-TAMRA-3′ (SEQ ID NO: 20). In addition, primers and probes corresponding to human glyceraldehyde-3-phosphate dehydrogenase (GAPDH) were also designed and synthesized, forward primer 5′-CAAGGTCATCCATGACAACTTTG-3′; reverse primer 5′-GGCCATCCACAGTCTTCTGG-3′; TAQMAN Probe 5′-FAM-ACCACAGTCCATGCCATCACTGCCA-TAMRA-3′.

Reverse transcription (RT) reactions were carried out in 50 ml reactions containing 1× TAQMAN RT buffer, 5.5 mM of magnesium chloride, 500 mM of each of the dNTPs, 2.5 mM of random hexamers, 0.4 U/ml of RNase inhibitor, 1.25 U/ml of multiscribe transcriptase, and 1 mg of RNA in RNase-free water. The RT reactions were performed at 42° C. for 60 minutes followed by a 15-minute heat inactivation step at 70° C. All RT products were treated with RNase H (2 unit/ml) for 20 minutes at 37° C. Samples were stored at −20° C.

PCR reactions were performed in 50 ml volume containing 1× TAQMAN buffer A, 5.5 mM of magnesium chloride, 200 mM of dATP, dCTP, dGTP, and 400mM of dUTP, 450 nM of the probe, 900 nM of each of the primers, 0.01 U/ml of AmpErase UNG, 0.025 U/ml of AmpliTaqGold DNA Polymerase, and 2 ng of cDNA in Rnase-free water. All PCR reagents were obtained from Applied Biosystems, Foster City, Calif.). The PCR reaction was then performed at 50° C. for 2 minutes followed by 95° C. for 10 min and then 40 cycles of 95° C. for 15 seconds and 60° C. for 1 minute. PCR reaction was conducted in an ABI Prism 7900 Sequence Detection System (SDS) (Applied Biosystems, Foster City, Calif.) and the results were analyzed using SDS program and expressed as a ratio to relative GAPDH levels as outlined in the ABS User Bulletin #2 (updated 10/2001) (Table 5).

Gene expression levels of CNGH0002 in endothelial cells cultured under different oxygenation conditions were quantitatively determined by TAQMAN analysis. An approximately 3-fold increase was detected in endothelial cells cultured in hypoxic conditions for 24 hours, similar to what was observed in hypoxic cancer cells. A decrease of gene expression occurred immediately following reoxygenation (Table 6). Therefore, CNGH0002 gene expression is not only regulated by hypoxia in cancer cells, but also in endothelial cells. Discovery of the association of the expression of the gene in these two cell types and under the influence of ambient dissolved oxygen levels thus make it a suitable target for both anticancer and antiangiogenesis therapies.

TABLE 5
CNGH0002 gene expression determined by quantitative real-time PCR

| Human Breast Carcinoma Cells | mRNA Copy Number | Relative |
|---|---|---|
| (MDA-MB231) | (Relative to GAPDH) | to Normoxia |
| Normoxia | 750.00 | 1.00 |
| Hypoxia 4 hr | 1346.08 | 1.75 |
| Hypoxia 24 hr | 1654.83 | 2.21 |
| Reoxia 4 | 906.26 | 1.21 |

TABLE 6
CNGH0002 gene expression determined by quantitative real-time PCR

| Human Micro Vascular Endothelial | mRNA Copy Number | Relative to |
|---|---|---|
| Cells (HMVEC) | (Relative to GAPDH)l | Normoxia |
| Normoxia | 2110.71 | 1.00 |
| Hypoxia 24 hr | 6198.43 | 2.94 |
| Reoxia 24 hr | 2345.48 | 1.11 |

Similarly, gene expression levels of CNGH0002 in normal human colon tissues or colon cancer tissues were quantitatively determined by TAQMAN analysis using RNA extracted from these tissues (Clontech, Palo Alto, Calif.). As shown in Table 7, a 5-fold increase in CNGH0002 was detected in colon cancer as compared to that in normal colon.

TABLE 7
CNGH0002 gene expression by quantitative real-time PCR

| | mRNA Copy Number | |
|---|---|---|
| Human colon tissue | (Relative to GAPDH) | Relative to Normal |
| Normal colon | 22.7 | 1.00 |
| Tumor colon | 109 | 4.80 |

1. An isolated nucleic acid molecule having a nucleotide sequence which is at least 95% identical to the nucleotide sequence of one of SEQ ID NOS: 1 and 4, the nucleic acid molecule encoding a protein having an amino acid sequence that is at least 95% identical to one of SEQ ID NOS: 2, 3 and 5.

2. A nucleic acid molecule comprising at least 45 nucleotide residues and having a nucleotide sequence identical to at least 45 consecutive nucleotide residues of one of SEQ ID NOS: 1 and 4, or an exact complement thereof.

3. A nucleic acid molecule which encodes a polypeptide having an amino acid sequence comprising one of SEQ ID NOS: 2, 3 and 5, or an exact complement thereof.

4. A nucleic acid molecule which encodes a fragment of a polypeptide having an amino acid sequence comprising one of SEQ ID NOS: 2, 3 and 5, wherein the fragment has an amino acid sequence comprising at least 15 consecutive amino acid residues of one of SEQ ID NOS: 2, 3 and 5.

5. A nucleic acid molecule which encodes a naturally occurring allelic variant of a polypeptide having an amino acid sequence comprising one of SEQ ID NOS: 2, 3 and 5.

6. A nucleic acid molecule which hybridizes with a nucleic acid molecule having the nucleotide sequence of one of SEQ ID NOS: 1 and 4, or an exact complement thereof in 6× sodium chloride/sodium citrate (SSC) at about 45° C. followed by washing in 0.2×SSC, 0.1% SDS at 65° C.

7. The nucleic acid molecule of claim 1, wherein the nucleic acid sequences further comprises a portion encoding a heterologous polypeptide.

8. The nucleic acid molecule according to any of claims claim 1-7, further comprising vector nucleic acid residues.

9. A host cell which contains the nucleic acid molecule according to any of claims claim 1-7

10. The host cell of claim 9 which is a mammalian host cell.

11. A kit comprising (i) a compound which hybridizes with a nucleic acid molecule of claim 1 in 6×SSC at about 45° C. followed by washing in 0.2×SSC, 0.1% SDS at 65° C., and (ii) instructions for use.

12. At least one CNGH0002 polypeptide, comprising all of the contiguous amino acids of at least one of SEQ ID NOS: 2, 3 and 5.

13. At least one CNGH0002 polypeptide, comprising at least 15 contiguous amino acids of at least one of SEQ ID NOS: 2, 3 and 5.

14. At least one CNGH0002 polypeptide, comprising at least one extracellular, transmembrane or cytoplasmic domain of at least one of SEQ ID NOS: 2, 3 and 5.

15. At least one CNGH0002 polypeptide, comprising at least one polypeptide having at least 90-99% identity to an amino acid sequence comprising all of the contiguous amino acids of at least one of SEQ ID NOS: 2, 3 and 5.

16. At least one CNGH0002 polypeptide, comprising at least one polypeptide having at least 99-99% identity to an amino acid sequence comprising at least 45 of the contiguous amino acids of at least one of SEQ ID NOS: 2, 3 and 5.

17. At least one CNGH0002 polypeptide, comprising at least one polypeptide encoded by at least one polynucleotide that hybridizes under stringent conditions to all of the contiguous nucleotides of at least one of SEQ ID NOS: 1 or 4 or a polynucleotide complementary thereto.

18. At least one CNGH0002 polypeptide, comprising at least one polypeptide encoded by at least one polynucleotide that hybridizes under stringent conditions to at least 45 of the contiguous nucleotides of at least one of SEQ ID NOS: 1 or 4 or a polynucleotide complementary thereto.

19. At least one CNGH0002 polypeptide, comprising 1-50 amino acid substitutions of at least one of SEQ ID NOS: 2, 3 and 5.

20. A CNGH0002 polypeptide according to any of claims 12-19, wherein said polypeptide has at least one activity of at least one CNGH0002 polypeptide.

21. A CNGH0002 antibody, comprising a monoclonal or polyclonal antibody, immunoglobulin fusion protein, or fragment thereof, that specifically binds at least one CNGH0002 polypeptide according to any of claims 12-19.

22. A nucleic acid encoding at least one CNGH0002 antibody according to claim 21.

23. A vector comprising at least one isolated nucleic acid of claims 22.

24. A host cell comprising an isolated nucleic acid according to claim 22.

25. A method for producing at least one CNGH0002 polypeptide or CNGH0002antibody, comprising translating a nucleic acid according to any of claims 1-7 and 22, under conditions in vitro, in vivo or in situ, such that the CNGH0002 polypeptide or antibody is expressed in detectable or recoverable amounts.

26. A composition comprising at least one CNGH0002 nucleic acid, CNGH0002 polypeptide, or CNGH0002 antibody according to any of claims 1-7, 12-19, or 21.

27. A composition according to claim 27, wherein said composition further comprises at least one pharmaceutically acceptable carrier or diluent.

28. A composition according to claim 28, administered in combination with at least one composition comprising at least one compound, composition or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an opthalmic, otic or nasal drug, a topical drug, a nutritional supplement, a cytokine, or a cytokine antagonist.

29. A composition according to claim 28, in a form of at least one selected from a liquid, gas, or dry, solution, mixture, suspension, emulsion or colloid, a lyophilized preparation, a powder.

30. A method for diagnosing a CNGH0002 related condition in a cell, tissue, organ or animal, comprising, contacting or administering a composition comprising means for detection of at least one CNGH0002 nucleic acid, polypeptide or antibody according to any of claims 1-7, 12-19, or 21, in said cell, tissue, organ or animal.

31. A method for treating a CNGH0002 related condition in a cell, tissue, organ or animal, comprising administering to a patient a composition comprising an effective amount of at least one CNGH0002 nucleic acid, polypeptide or antibody according to any of claims 1-7, 12-19, or 21, with, or to, said cell, tissue, organ or animal.

32. A method for treating a CNGH0002 related condition in a cell, tissue, organ or animal, comprising administering to a patient a composition comprising an effective amount of at least one modulator of CNGH0002 nucleic acid or CNGH0002 polypeptide levels or a modulator of CNGH0002 activity in said cell, tissue, organ or animal.

33. A method of treating a hypoxia-related condition in a cell, tissue, organ or animal comprising administering to a patient a composition comprising an effective amount of at least one CNGH0002 nucleic acid, polypeptide or antibody according to any of claims 1-7, 12-19, or 21, with, or to, said cell, tissue, organ or animal.

34. A method of treating a hypoxia-related condition in a cell, tissue, organ or animal comprising administering to a patient a composition comprising an effective amount of at least one modulator of CNGH0002 nucleic acid or CNGH0002 polypeptide levels or a modulator of CNGH0002 activity in said cell, tissue, organ or animal.

35. A method according to any one of claims 31 or 33, wherein said effective amount is 0.001-50 mg of CNGH0002 antibody; 0.000001-500 mg of said CNGH0002 polypeptide; or 0.0001-100 μg of said CNGH0002 nucleic acid per kilogram of said cells, tissue, organ or animal.

36. A method according to any one of claims 31-34, wherein said administration is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

37. A method according to claim 36, further comprising administering, prior, concurrently or after said (a) contacting or administering, at least one composition comprising an effective amount of at least one compound or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional supplement, a cytokine, or a cytokine antagonist.

38. A device, comprising at least one isolated CNGH0002 polypeptide, antibody or nucleic acid according to any of claims 1-7, 12-19, or 21, wherein said device is suitable for contacting or administerting said at least one of said CNGH0002 polypeptide, antibody or nucleic acid, by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

39. An article of manufacture for human pharmaceutical or diagnostic use, comprising packaging material and a container comprising at least one isolated CNGH002 polypeptide, antibody or nucleic acid according to any of claims 1-7, 12-19, or 21.

40. The article of manufacture of claim 39, wherein said container is a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal delivery device or system.

41. A method for producing at least one isolated CNGH002 polypeptide, antibody or nucleic acid according to any of claims 1-7, 12-19, or 21, comprising providing at least one host cell, transgenic animal, or transgenic plant capable of expressing in detectable or recoverable amounts said polypeptide, antibody or nucleic acid.

42. At least one CNGH002 polypeptide, antibody or nucleic acid, produced by a method according to claim 41.

43. A fusion polypeptide comprising at least one CNGH0002 polypeptide according to claim 12 or a fragment thereof operably linked to a heterologous polypeptide.

44. A fusion polypeptide of claim 43 wherein the heterologous polypeptide is selected from a member of the immunoglobulin protein family or a fragment thereof.

45. A method for detecting the presence or absence of a polypeptide of claim 12 in a biological sample comprising obtaining a biological sample from a test subject and contacting the biological sample with a compound or an agent capable of detecting said such that the presence of a polypeptide or nucleic acid of the invention is detected in the biological sample.
